(19) United States
(12) Patent Application Publication
(10) Pub. No.: US 2026/0002906 A1
(43) Pub. Date: Jan. 1, 2026

(54) INTEGRATED METHOD FOR COMPOSITIONAL EVALUATION IN OIL WELLS

(71) Applicants: PETRÓLEO BRASILEIRO S.A. - PETROBRAS, Rio de Janeiro (BR); UNIVERSIDADE FEDERAL DE GOIÁS - UFG, Goiânia (BR)

(72) Inventors: Lidya Cardozo Da Silva, Goiânia (BR); Gabriel Henry Morais Dufrayer, Goiânia (BR); Joveilton Batista Da Silva Júnior, Goiânia (BR); Daniel Silva Dubois, Rio de Janeiro (BR); Ygor Dos Santos Rocha, Rio de Janeiro (BR); Luiz Henrique Keng Queiroz Júnior, Goiânia (BR); Boniek Gontijo Vaz, Goiânia (BR); Joelma Pimentel Lopes, Rio de Janeiro (BR); Jussara Valente Roque, Goiânia (BR); Hugo Gontijo Machado, Goiânia (BR)

(21) Appl. No.: 19/242,227

(22) Filed: Jun. 18, 2025

(30) Foreign Application Priority Data
Jun. 26, 2024 (BR) BR 1020240131088

(51) Int. Cl. G01N 27/623; G01N 1/38; G01N 1/44; G01N 33/28

(52) U.S. Cl. CPC G01N 27/623; G01N 1/38; G01N 1/44; G01N 33/2823

(57) ABSTRACT

The present invention relates to an integrated method to identify the reservoir compartmentalization and characterize samples of different compositional gradations and types of operation (PVT and DST) through the ESI (−) and APPI (+) FT-ICR-MS techniques in combination with the application of machine learning algorithms. In this way, the present invention presents an integrated method for compositional evaluation in oil wells to: (i) analyze the compositional variation of the polar components in reservoirs; (ii) study the molecular distribution in reservoirs with varied thicknesses; and (iii) explore the composition of the polar components as molecular indicators for understanding compartmentalization and lateral and vertical connectivity between the fluids in reservoirs.

### FIELD OF THE INVENTION

The present invention relates to an integrated method for compositional evaluation in oil wells to: (i) analyze the compositional variation of the polar components in reservoirs; (ii) study the molecular distribution in reservoirs with varied thicknesses; and (iii) explore the composition of the polar components as molecular indicators for understanding compartmentalization and lateral and vertical connectivity between the fluids in reservoirs. In this way, the present invention presents an integrated method for identifying the reservoir compartmentalization and characterizing samples of different compositional gradations and types of operation (PVT and DST) through the ESI (−) and APPI (+) FT-ICR-MS technique in combination with the application of machine learning algorithms.

### BACKGROUNDS OF THE INVENTION

The effective exploration and management of oil reservoirs require a deep understanding of the chemical composition of the fluids contained within (Peters K. E., Walters C. & Moldowan J. M. (2005). The Biomarker Guide. 2nd edn. Cambridge University Press, Cambridge), as well as the physical connectivity between wells (Walters. C. C. (2020). Organic geochemistry at varying scales: From kilometers to angstroms. Geological Society Special Publication. 484(1). 121-137. https://doi.org/10.1144/SP484.7).

The great contribution and success of organic geochemistry in minimizing risks and increasing efficiency in the development of the oil production is explained, in part, by the adoption and innovation in instrumentation and analytical developments (Dowey. P. J., Osborne. M., & Volk. H. (2020). Application of analytical techniques to petroleum systems: An introduction. Geological Society Special Publication. 484(1). 1-7. https://doi.org/10.1144/SP484-2020-57). It is through instrumentation and analytical protocols that the compositional information of fluids, sediments and rocks comes to light to be deciphered, retelling the geological history of an oil system, and to assist, minimizing risk, the economic development of geological prospects and plays. However, innovations and new protocols in analytical methods applied to the organic geochemistry are necessary in the current scenario of energy transition towards a low-carbon economy (Lopes. J. P., Rangel. M. D., Morais. E. T. de. & Aguiar. H. G. M. de. (2008). Geoquímica de reservatórios. Revista Brasileira de Geociências. 38(1). 03-18. https://doi.org/10.25249/0375-7536.2008381S0318).

Traditionally, geochemical analyses of oil and fluids use the saturated and aromatic hydrocarbon fractions (England. W. A. (2007). Reservoir geochemistry-A reservoir engineering perspective. Undefined. 58(3-4). 344-354. https://doi.org/10.1016/J.PETROL.2005.12.012). However, the polar components, which can correspond to up to 20% of the oil, have not been properly investigated due to analytical limitations. With the advent of the petroleomics, which aims at correlating and predicting the behavior, reactivity, and properties of oils and its derivatives from detailed composition data, this scenario has changed completely (Marshall. A. G., & Rodgers. R. P. (2004). Petroleomics: The Next Grand Challenge for Chemical Analysis. Accounts of Chemical Research. 37(1). 53-59. https://doi.org/10.1021/ar020177t; Rodrigues Covas. T., Santos de Freitas. C., Valadares Tose. L., Valencia-Dávila. J. A., dos Santos Rocha. Y., Duncan Rangel. M., Cabral da Silva. R., & Gontijo Vaz. B. (2020). Fractionation of polar compounds from crude oils by hetero-medium pressure liquid chromatography (H-MPLC) and molecular characterization by ultrahigh resolution mass spectrometry. Fuel. 267. 117289. https://doi.org/10.1016/J.FUEL.2020.117289). Through the Fourier Transform Mass Spectrometry (FT-MS) technique, the molecular formulas of thousands of polar components of crude oil and its derivatives can be determined and thus ordered into their most varied classes: N, NO, NS, O₂, and related classes, and also according to their degrees of unsaturation (DBE, double bond equivalent) and their carbon numbers (Marshall. A. G., & Rodgers. R. P. (2004). Petroleomics: The Next Grand Challenge for Chemical Analysis. Accounts of Chemical Research. 37(1). 53-59.https://doi.org/10.1021/ar020177t). Oil from different origins, biodegradation levels and thermal evolution have presented quite distinct and characteristic profiles in FT-MS analysis (Rocha. Y. dos S., Pereira. R. C. L., & Mendonça Filho. J. G. (2018). Negative electrospray Fourier transform ion cyclotron resonance mass spectrometry determination of the effects on the distribution of acids and nitrogen-containing compounds in the simulated thermal evolution of a Type-I source rock. Organic Geochemistry. 115. 32-45. https://doi.org/10.1016/J.ORGGEOCHEM.2017.10.004; Vaz B. G., Silva. R. C., Klitzke. C. F., Simas. R. C., Lopes Nascimento. H. D., Pereira. R. C. L., Garcia. D. F., Eberlin. M. N., & Azevedo. D. A. (2013). Assessing biodegradation in the llanos orientales crude oils by electrospray ionization ultrahigh resolution and accuracy Fourier transform mass spectrometry and chemometric analysis. Energy and Fuels. 27(3). 1277-1284. https://doi.org/10.1021/EF301766R). Thus, these can be targeted to reflect distinct variations by compound classes according to specific characterization interests. Therefore, FT-MS can be used in the comprehensive characterization of oil and derivatives, and the results obtained can be used to support exploration and production, refining, distribution and SEH (Safety, Environment and Health) activities (Dalmaschio. G. P., Malacarne. M. M., de Almeida. V. M. D. L., Pereira. T. M. C., Gomes. A. O., de Castro. E. V. R., Greco. S. J., Vaz. B. G., & Romão. W. (2014). Characterization of polar compounds in a true boiling point distillation system using electrospray ionization FT-ICR mass spectrometry. Fuel. 115. 190-202. https://doi.org/10.1016/J.FUEL.2013.07.008; Hughey. C. A., Hendrickson. C. L., Rodgers. R. P., & Marshall. A. G. (2001). Elemental composition analysis of processed and unprocessed diesel fuel by electrospray ionization Fourier transform ion cyclotron resonance mass spectrometry. Energy and Fuels. 15(5). 1186-1193. https://doi.org/10.1021/EF010028B; Smith. D. F., Schaub. T. M., Rahimi. P., Teclemariam. A., Rodgers. R. P., & Marshall. A. G. (2007). Self-association of organic acids in petroleum and Canadian bitumen characterized by low- and high-resolution mass spectrometry. Energy and Fuels. 21(3). 1309-1316. https://doi.org/10.1021/EF060387C/SUPPL_FILE/EF060387CSI200611 08_041320.GIF).

The compositional heterogeneity of fluids (water, oil and gas) in the reservoir, on a vertical and lateral scale, is used in reservoir engineering strategies to support practical actions and minimize risks in the exploration, production and development of oil fields. These reflect geological aspects on a regional and reservoir scale. In this way, the study of these heterogeneities can be used not only as a descriptive tool for the reservoir, but also to delimit an accumulation and for regional exploration (Lopes. J. P., Rangel. M. D., Morais. E. T. de. & Aquíar. H. G. M. de. (2008). Geoquímica de reservatórios. Revista Brasileira de Geociências. 38(1). 03-18. https://doi.org/10.25249/0375-7536.2008381S0318).

The compositional differences found in oil are due to differences in the organic matter sedimented in the source rock. The sedimented organic matter comprises several biopolymers that are converted into kerogen during the diagenesis. Kerogen is the insoluble part of the organic matter that is converted into bitumen during the maturation process. Bitumen is the extractable part, mostly composed of heavy hydrocarbons.

Bitumen transforms into oil during the migration processes, in which lighter hydrocarbons migrate more easily. Oil, then, is the liquid organic substance recovered in the wells. In turn, the liquid expelled from the source rock varies in relation to its composition and the time of expulsion.

Similarly, a source rock may contain organic matter of varied composition. Therefore, the variation in the organic matter of origin and the process of expulsion of the liquid are related to the compositional variety of oils found in the reservoirs. However, once a reservoir is filled, the compositional variations are eliminated by density-driven forces and molecular diffusion mechanisms until the chemical and mechanical equilibrium is reached (Lopes. J. P., Rangel. M. D., Morais. E. T. de. & Aguíar. H. G. M. de. (2008). Geoquímica de reservatórios. Revista Brasileira de Geociências. 38(1). 03-18. https://doi.org/10.25249/0375-7536.2008381S0318).

The molecular diffusion plays a critical role in the reservoir system transitioning towards an equilibrium state (Yang. Y., Stenby. E. H., Shapiro. A. A., & Yan. W. (2022). Diffusion Coefficients in Systems Related to Reservoir Fluids: Available Data and Evaluation of Correlations. Processes. 10(8). https://doi.org/10.3390/pr10081554). Similarly, forces driven by density differences between the reservoir fluids tend to promote a homogeneous oil column. Thus, the fluids in a reservoir only remain heterogeneous if there is a barrier that isolates them (Lopes. J. P., Rangel. M. D., Morais. E. T. de. & Aguiar. H. G. M. de. (2008). Geoquímica de reservatórios. Revista Brasileira de Geoci\hat{e}ncias. 38(1). 03-18. https://doi.org/10.25249/0375-7536.2008381S0318).

In this scenario, the evaluation of the composition of the reservoir fluids on a spatial and temporal scale is one of the main tasks of the reservoir geochemistry. In addition, the evaluation of the composition of oils at the molecular level can reveal compartmentalized regions in the studied fields, and thus, can be used in the evaluation of the reservoir continuity (Vaz. B. G., Silva. R. C., Klitzke. C. F., Simas. R. C., Lopes Nascimento. H. D., Pereira. R. C. L., Garcia. D. F., Eberlin. M. N., & Azevedo. D. A. (2013). Assessing biodegradation in the llanos orientales crude oils by electrospray ionization ultrahigh resolution and accuracy Fourier transform mass spectrometry and chemometric analysis. Energy and Fuels. 27(3). 1277-1284. https://doi.org/10.1021/EF301766R). That said, the following challenges arise:
1. Connectivity between Wells: One of the central challenges in reservoir management is determining whether different wells are interconnected. This connectivity has direct implications for production, since interconnected wells can drain the same portion of the reservoir. The chemical composition of the oil can provide valuable clues in this regard, since connected wells tend to have similar compositions.
2. Compositional Gradation in Relation to Depth: As one goes deeper into a reservoir, it is common to observe variations in the composition of the oil. These gradations can be influenced by factors such as temperature, pressure and rock-fluid interactions. The detailed understanding of this vertical compositional variation is crucial to optimizing production strategies and understanding the geological history of the reservoir.
3. Collection Methods and Their Implications: The way in which oil is collected can significantly influence its apparent composition. Cased-hole formation tests, which evaluate the composition directly in the well before the complete installation of the equipment, can provide a more reliable compositional profile of the reservoir in question. On the other hand, production tests, which evaluate oil already in the production phase, can present compositional alterations due to the mixtures of different zones of the reservoir or production effects.

In short, the characterization and management of oil reservoirs are multidimensional issues that require scientific rigor and an integrated approach. The composition of the oil, in its complexity, offers a portal to understand the geological history, the connectivity between wells, the compositional gradation in relation to depth and the impacts of the recovery techniques. To navigate this maze of information and challenges, advanced analytical techniques are required, from petroleomics (Lopes. J. P., Rangel. M. D., Morais. E. T. de. & Aguíar. H. G. M. de. (2008). Geoquímica de reservatórios. Revista Brasileira de Geociências. 38(1). 03-18. https://doi.org/10.25249/0375-7536.2008381S0318) to computational approaches such as machine learning, as well as a holistic understanding of geology, geochemistry and reservoir engineering.

As can be seen below, the state of the art does not present the proposed solution of the present invention of a more robust integrated method to identify the reservoir compartmentalization and characterize samples of different compositional gradations and types of operation (PVT and DST) through the ESI (−) and APPI (+) FT-ICR-MS technique together with computational approaches such as machine learning.

### STATE OF THE ART

Although document US 2023/0175369 aims at advancing the analysis of oil reservoir fluids, the present invention is distinguished by its unique focus and advanced methodologies, providing deep insights and direct practical applications for the oil industry. In particular, the present invention resides in the detailed analysis s of the polar components of the oil, using the FT-ICR MS technique, complemented by the application of machine learning algorithms, which allows a differentiated and more accurate characterization of the oil samples.

Contrary to what is observed in this American document, which does not specify the use of Fourier Transform Ion Cyclotron Resonance Mass Spectrometry (FT-ICR MS) nor does it detail ionization techniques such as ESI (Electrospray Ionization) and Atmospheric Pressure Photoionization (APPI), nor collection methods such as PVT (Pressure-Volume-Temperature) and DST (Cased-hole drill-stem test), the present invention deepens into these techniques and uses machine learning models such as PLS-DA (Partial Least Squares Discriminant Analysis) to explore the molecular composition of the samples. This approach allows not only to focus on the oil production, but also to differentiate collection methods and geological formations, directly connecting theory to practice.

In this way, the specificity of the present invention brings clear advantages over the American document, such as:
The characterization of the polar components of the oil exclusively through FT-ICR MS analyses coupled to the two ionization sources ESI (−) and APPI (+), the results of which are essential for understanding the complexity and chemical diversity of the oil; and
The integration of machine learning with FT-ICR MS in the present invention allows advanced analyses and interpretations of large compositional data sets, which was not mentioned in the American document.

In addition, the differentiation between PVT and DST samples at the molecular level with the FT-ICR MS technique illustrates a notable advance, providing a deeper understanding of the properties and behaviors of the oil reservoir fluids. This advanced and detailed methodology reinforces the practical relevance of the invention, offering a unique contribution, going beyond the generalizations presented in the American document. Therefore, the present invention presents a more robust methodological approach and is directly applicable to the oil industry.

The study titled “Combining biomarker and bulk compositional gradient analysis to evaluate reservoir connectivity” conducted by Pomerantz et al., 2010, used the ESI (−) FT ICR MS technique to analyze three samples from different depths collected from the same well. Although the samples showed similarity, suggesting that the reservoir is in communication in this well, the differences observed in the composition of the acyclic/cyclic O₂ class, identified through the GCxGC analysis, indicated different oil pulses, the first of which was severely biodegraded. It is important to emphasize that the differences observed in the compositions of the oils did not come from the ESI(−)FT-ICR MS analysis but from the optical spectroscopy and GCxGC (Comprehensive Two-Dimensional Gas Chromatography) analyses. Through the FT-ICR MS technique, it was found that the samples were very similar, which indicated that there was no barrier to the flow of fluids in the reservoir.

However, the present invention addresses to a significant set of data, involving 88 oil samples from 27 wells in a given production field, that is, the characterizations performed encompassed an extensive reservoir, both in area and thickness. The samples were collected by cable tests (PVT chambers) and cased well tests (DST). Two ionization sources coupled to the FT-ICR MS were used, the ESI (−) that ionizes the most polar compounds in the oil and the APPI (+) that ionizes compounds of medium polarity, not ionized by ESI, such as sulfur compounds and hydrocarbons. Both techniques can ionize compounds of high molecular weight that are not ionized by traditional techniques such as GCMS or GC-GC. In addition to using the two ionization techniques to evaluate the data, the spectra were obtained in triplicate to have greater analytical reliability. With this, a large set of data was generated.

Given the large number of variables resulting from the analysis, multivariate analysis was used to filter those that had the greatest weight in explaining the model, both for the compositional gradation and the reservoir compartmentalization. To differentiate the types of operation, PVT and DST, the models were able to correctly classify the samples, achieving 100% accuracy.

Therefore, the present invention presents a robust method to identify reservoir compartmentalization and characterize samples of different compositional gradations and types of operation (PVT and DST) through the ESI (−) and APPI (+) FT-ICR-MS technique.

The paper “Using machine learning-based variable selection to identify hydrate related components from FT-ICR MS spectra” describes the use of machine learning techniques to select variables based on FT-ICR MS spectra with the aim of identifying components related to hydrate formation in crude oils. The study focuses on solving a specific problem in the oil and gas industry, which is the agglomeration of hydrates causing blockages in pipelines.

The present invention significantly differs from the scope and methodologies discussed in this document, particularly due to its diversified and innovative approach to the study of the composition of crude oils. While this document is limited to the analysis of samples from a single location in a Norwegian reservoir, the investigation herein encompasses a matrix of samples collected from 26 wells distributed in 12 production modules, encompassing 4 geological formations in Brazil. This geological and operational diversity allows for a richer and more contextualized comparative analysis, transcending the limitations of studies based on a single type of sample or location.

In addition, complementary analytical techniques were employed, such as ESI (−) for high polarity compounds and APPI (+) for medium polarity compounds, broadening the spectrum of compounds analyzed compared to the single ESI (+) approach used in the document under discussion. This methodological duality significantly enriches the understanding of the crude oil composition, offering a holistic view that goes beyond the analytical capabilities of a single ionization technique. The present invention deepens the exploratory analysis through the use of a variety of graphical tools, such as class diagrams, DBEs, carbon number and ternary diagrams. This approach not only facilitates the visual interpretation of complex data, but also surpasses the spectral overlap and subtraction methodology found in the document in question, allowing a detailed and targeted investigation of the composition of the crude oils.

In the dimension of machine learning models, the elaboration of 22 PLS-DA models, each meticulously adjusted to reflect the particularities of the crude oil samples, demonstrates a level of complexity that goes beyond that explored in the aforementioned document. This detailed approach allows capturing specific nuances related to the collection techniques, compound classes and geochemical characteristics, substantially differentiating the present analysis. The selection of variables by the OPSDA method and the subsequent optimization of ratios between compounds, highlighting the differences between data sets, represents a methodological innovation relation to the VIP-Score approach of the aforementioned document. This strategy not only highlights distinctions between sample groups more effectively, but also underlines the innovative character of the present invention.

Focusing on samples from a Brazilian reservoir known for its geological complexity and strategic importance, the present invention offers valuable insights for petroleomics science and practical applications for the oil industry. This integration between theory and practice highlights the uniqueness, relevance and applicability of the invention in oil exploration and production contexts, significantly differentiating the same from the generalizations and more limited approaches discussed in the context of the document in question. Therefore, by combining a comprehensive and differentiated methodology with a specific and relevant application, the present invention not only advances knowledge in the field of petroleomics, but also offers practical and innovative solutions that directly respond to the needs of the oil industry, establishing itself as a significant milestone in the evolution of oil composition analysis and underlining its fundamental distinction in relation to the study mentioned in the aforementioned document.

The paper “When Petrophysics Meets Big Data: What can Machine Do?” is a comprehensive review that discusses the intersections between petrophysics, big data, machine learning, and artificial intelligence, highlighting the potential of these technologies to transform petrophysical data analysis. While the paper provides valuable insight into the theoretical and practical applications of machine learning (ML) and artificial intelligence (AI) in the processing and interpretation of a wide range of petrophysical data, its general and theoretical approach differs significantly from the specific and practical focus of the present invention.

The present invention is grounded on specific data collection, rigorous experimentation, and generation of concrete results, focusing on the detailed analysis of the compositional variation of polar components in oil reservoirs to evaluate inter-reservoir connectivity and compositional gradation. While the aforementioned document suggests an overview of how ML and AI can be explored to address to challenges in petrophysics in a broad way, the present invention presents a specific application, developing an innovative method that advances the solution of specific problems related to the polar composition in reservoirs. This contrast between the theoretical and generalized nature of the review paper and the targeted and experimental approach of the present invention stands out in the practical applicability of the invention. Therefore, despite the contributions of the aforementioned review document to the general understanding of the field, the present invention stands out for its specific and tangible contribution to the compositional analysis of reservoirs, offering practical and innovative methodologies.

### SUMMARY OF THE INVENTION

The invention described aims at analyzing and understanding the composition of oil reservoirs, especially the compositional variation of the polar components. Its application potential is vast and can thus revolutionize several aspects of the oil and gas industry. Some potential applications include:
1. Production Optimization: By understanding the connectivity between the well fluids through the composition of the polar components, companies can optimize their production, avoiding excessive drainage of a given section of the reservoir and ensuring that resources are extracted in a balanced manner.
2. Effective Exploration: The detailed analysis of oil composition, especially in relation to depth, can help companies identify reservoir zones that are richer in oil or gas, making exploration more efficient and reducing costs.
3. Prediction of the Oil Quality: By understanding the compositional variation and how different factors, such as recovery techniques and depth, influence this variation, companies can predict the quality of the oil they are producing. This can influence decisions about refining and commercialization.
4. Development of New Technologies: Detailed information about the oil composition can lead to the development of new technologies or chemicals for treating, separating and refining oil.
5. Reduction of Environmental Impacts: By optimizing production and recovery, the need to drill additional wells or use more invasive techniques can be reduced, which can minimize the environmental impacts.
6. Data-Driven Decision Making: Combining detailed oil composition analysis with advanced techniques such as machine learning can enable more informed, data-driven decision making, leading to more efficient and profitable operations.

Therefore, the application of this invention can broadly benefit the oil industry, from the exploration phase to production, refining and commercialization. By providing a deeper understanding of the composition of the reservoirs and how it is influenced by various variables, this invention has the potential to boost the efficiency, profitability and sustainability of the industry.

Therefore, the present invention presents a more robust methodological approach and directly applicable to the oil industry through the integrated method for compositional evaluation in oil wells comprising the characterization of the samples of different compositional gradations and types of operation (PVT and DST) through the ESI (−) and APPI (+) FT-ICR-MS technique in combination with the application of machine learning algorithms.

### BRIEF DESCRIPTION OF THE FIGURES

The present invention will be described below, with reference to the attached FIGS. 1 to 29 that, in a schematic manner and not limiting the inventive scope, represent examples of its embodiment.

FIG. 1 represents the Distribution of classes of the ESI (−) FT-ICR MS of 88 samples in triplicate, in which the dotted red line shows the 1% limit.

FIG. 2 shows the Histograms of the frequency of occurrence of the individual classes of the ESI (−) FT-ICR MS of 88 samples in triplicate.

FIG. 3 shows the Histogram of the coefficient of variation (CV) for the selected classes.

FIG. 4 represents the Distribution of classes of the APPI (+) FT-ICR MS of 88 samples in triplicate, in which the dotted red line shows the 1% limit and (+) indicates that the class is protonated and (·) that it is radical.

FIG. 5 shows the Histograms of the frequency of occurrence of the individual APPI (+) FT-ICR MS classes of 88 samples in triplicate.

FIG. 6 shows the Histogram of the coefficient of variation (CV) for the selected classes.

FIG. 7 represents the Distribution of (A) carbon number and (B) DBEs in relative abundance for the ESI (−) data considering the sum of the classes N, N₂, NO, O and O₂.

FIG. 8 represents the Distribution of (A) carbon number and (B) DBEs in relative abundance for the APPI (+) data considering the sum of the HC, N, NO, O, OS and S classes.

FIG. 9 represents the (A) Distribution of classes and (B) distribution of minority classes for the ESI (−) data.

FIG. 10 represents the (A) Distribution of classes and (B) distribution of minority classes for the APPI (+) data.

FIG. 11 illustrates the Ternary diagram of the distribution of classes for the ESI (−) data, in which in the first diagram the classes N, NO and O₂ and in the second the classes O₂, O and N₂.

FIG. 12 illustrates the Ternary diagram of the distribution of classes for the APPI (+) data, in which the first diagram shows the classes St, S. and Ost, the second one shows the classes N., NO. and NO⁺ and the third one shows the classes HC⁺, HC. and O.

FIG. 13 illustrates the model reaction of oxidation of indene (C₉H₈).

FIG. 14 shows the Gibbs' Potential Energy Surface for the oxidation reaction of indene (C₉H₈); the energies are in kJ/mol.

FIG. 15 illustrates Scores and loadings of the PCA of the (A) ESI (−) and (B) APPI (+) FT-ICR MS data of 88 samples categorized by the type of operation: DST, PVT and PVT-C.

FIG. 16 shows the Operation Type Prediction of the ESI (−) and APPI (+) variable classes for PVT (•, blue dot) and DST (•, red dot) samples; in which the dotted line indicates the threshold.

FIG. 17 illustrates the Radar of the optimized ratios for the general set of 88 samples analyzed by (A) ESI (−) and (B) APPI (+) FT-ICR MS.

FIG. 18 shows the Distribution of (A) carbon numbers and (B) DBEs in relative abundance for the ESI (−) data considering the sum of the classes N, N₂, NO, O and O₂.

FIG. 19 shows the distribution of (A) carbon number and (B) DBEs in relative abundance for the APPI (+) data considering the sum of the HC, N, NO, O, OS and S classes.

FIG. 20 shows the (A) Class distribution and (B) minority class distribution for the ESI (−) data.

FIG. 21 shows the (A) Class distribution and (B) minority class distribution for the APPI (+) data.

FIG. 22 shows the ternary diagram of the class distribution for the ESI (−) data, in which the first diagram shows the N, NO and O₂ classes and the second diagram shows the O₂, O and N₂ classes, highlighting the formations.

FIG. 23 shows the ternary diagram of the class distribution for the APPI (+) data, in which the first diagram shows the classes St, S. and Ost, the second diagram shows the classes N·, NO· and NO⁺ and the third diagram shows the classes HC⁺, HC· and O, highlighting the formations.

FIG. 24 illustrates PCA scores and loadings of the (A) ESI (−) and (B) APPI (+) FT-ICR MS data of 83 samples categorized by geological formation B and C.

FIG. 25 shows the Prediction of the geological formation of the ESI (−) and APPI (+) variable classes for samples from formation B (•, blue dot) and formation C (•, red dot), in which the dotted line indicates the threshold.

FIG. 26 shows the Radar of the optimized ratios to differentiate the geological formation of the overall set of 83 samples analyzed by (A) ESI (−) and (B) APPI (+) FT-ICR MS.

FIG. 27 illustrates the (A) Scores, (B) PCA loadings and (C) fluid connectivity model for samples from module 5.

FIG. 28 illustrates the Representation of fluid connectivity in the reservoir: lateral and vertical analysis between the wells of each module.

FIG. 29 illustrates the flowchart of the entire process of sample preparation, data processing in petroleomics and data processing to generate valuable insights.

### DETAILED DESCRIPTION OF THE INVENTION

The proposed invention addresses some of the main issues and challenges related to the management of oil reservoirs and the chemical composition of the oil. Thus, the invention can solve or minimize the difficulties as follows:
1. Connectivity between By analyzing the Wells: compositional variation of the polar components in different reservoirs and exploring the composition of the polar components as molecular indicators, it is possible to obtain more accurate information about the connectivity between wells. If the polar components of two wells are highly similar, this may indicate a strong connectivity between the same.
2. Compositional Gradation with Relation to Depth: By investigating changes in the polar composition and studying the molecular distribution in reservoirs of different thicknesses, the invention provides more detailed insights into how the composition of the oil varies with depth. This is crucial for understanding the structure and geological history of the reservoir.
3. Collection Methods and Their Implications: With a focus on the compositional analysis of the polar components, the invention is applicable to differentiating between compositions originating from different collection methods or mixtures from different zones of the reservoir.

The invention provides tools and methods to better understand the complex chemical composition of fluids in oil reservoirs. By focusing on specific components, such as polar components, the invention provides a more detailed and focused approach to address to critical issues in the reservoir management. This approach, combined with advanced analytical techniques and a holistic understanding of the relevant fields, has the potential to significantly improve the effectiveness of the oil reservoir exploration and management.

Thus, the present invention presents an integrated method to identify the reservoir compartmentalization and characterize samples of different compositional gradations and types of operation (PVT and DST) through the ESI (−) and APPI (+) FT-ICR-MS technique in combination with the application of machine learning algorithms.

The proposed method aims at deepening the understanding of the composition of the oils extracted from different oil wells and to evaluate the connectivity of the fluids between these wells. Petroleomics plays a fundamental role in the characterization of the reservoir fluids, providing crucial information on the molecular composition of oil samples and providing valuable insights into the reservoir geochemistry.

As will be observed in the examples section, the results obtained in this invention are presented in a sequential and methodological manner, reflecting the complexity and depth of the analysis performed in the reservoir X. The example below will demonstrate the analytical evaluation of the 88 samples (Table 5), based on the application of the ESI (−) and APPI (+) FT-ICR MS techniques, which allowed the collection of detailed data on the chemical composition of the samples from different modules, wells and geological formations (A, B, C and D).

The example sequentially presents the reliability analysis of the triplicates, emphasizing the use of the coefficient of variation to select the most appropriate classes of heteroatoms. This step ensures the consistency and reliability of the data, which are essential for the subsequent analysis. The example then moves on to present the results related to the collection methods (PVT and DST), illustrating how each approach influences the chemical composition of the oil samples, followed by the analysis of the samples based on the characteristics of the geological formations A, B, C and D, revealing distinct compositional patterns and insights into the geological influence on the composition of the oil.

The example concludes with a detailed investigation of the fluid connectivity between the wells in the reservoir, analyzing how the chemical composition reflects the interaction and sharing of the fluids at different locations. This part is crucial to understanding the dynamics of the reservoir and how the wells influence each other in terms of production and fluid extraction. Through this logical and detailed progression in the presentation of the results, it was possible to unravel the compositional complexities and the interconnections of the fluids in the reservoir X, providing a solid basis for future management and exploration decisions.

### Example of Embodiment/Tests and Results

The components of the invention are mainly related to the process described below, from sample preparation to data interpretation and analysis, which are:

### 1) Oil Samples:

88 oil samples.
Samples collected by the types of operation: PVT and DST.
Samples from 4 geological formations (A, B, C and D).

### 2) Reagents and Solutions:

Toluene: for dissolving crude oil.
Methanol: used in the preparation of the analysis solution.
NH₄OH (ammonium hydroxide): added to the analysis solution.
Na trifluoroacetate (NaTFA): calibration solution.
Other HPLC grade solvents, such as acetonitrile and dichloromethane, for example.

### 3) Main Equipment:

FT-ICR MS 7T SolariX 2xR: mass spectrometry equipment produced by Bruker Daltonics, coupled to the Electrospray (ESI) and Atmospheric Pressure Photoionization (APPI) source.

### 4) Software:

DataAnalysis (Bruker): used to recalibrate the raw mass spectra.
Composer 64 Version 1.5.3 (Sierra Analytics Inc): for assigning molecular formulas. Other commercial software may be used.
Software developed at LaCEM Laboratory of Chromatography and Mass Spectrometry of the Federal University of Goiás (UFG), used for calculating spectral noise, alignment and other data processing. Other commercial software may be used.
Thanus (LaCEM-UFG): Software developed at LaCEM, for visualization and interpretation of data. Other commercial software may be used.
Matlab 2020a (MathWorks Inc) and Python: for multivariate data analysis. Other commercial software may be used.

### 5) Procedures:

Obtaining crude oil samples through PVT and DST operations from the well drilling step, where PVT samples are collected prior to DST;
Sample preparation: dissolution, dilution and addition of reagents;
Coupling of the FT-ICR MS equipment to the ESI (−) or APPI (+) ionization sources, depending on the desired analysis;
Calibration of the equipment.
Acquisition of spectra in triplicate.
Recalibration of the spectra and subsequent assignment of molecular formulas.
Alignment of data and processing of the triplicates.
Visualization and interpretation of the results of the FT-ICR MS analyses.
Multivariate analysis.

Briefly, the crude oil samples were prepared by dissolving 10 mg of oil in 10 mL of toluene. For ESI (−) analyses, 500 μL of the stock solution were collected and transferred to a vial containing 500 μL of methanol. 50 μL of NH₄OH were added to this solution. The final concentration of oil in the analysis solution was 500 ppm in toluene/methanol (50:50) and 5.0% NH₄OH. The solvents methanol, toluene and ammonium hydroxide were of HPLC grade and purchased from J. T. Baker (Phillipsburg, NJ, USA). For APPI (+) analyses, the toluene solution of the samples was directly injected into the mass spectrometer.

Mass spectrometry analyses were performed using a FT-ICR MS 7T SolariX 2xR (Bruker Daltonics-Bremen, Germany) equipment coupled to the ESI or APPI source. The equipment was calibrated daily with a 0.1 μL·mL⁻¹ solution of the sodium trifluoroacetate (NaTFA) calibrant from Sigma-Aldrich (Steinhein, Germany), for positive and negative modes, in the m/z range from 150 to 2000. The average calibration error ranged from 0.02 to 0.04 ppm in linear regression mode. 8MW data sets were acquired through magnitude mode with the detection range of m/z 150-2000.

In order to ensure data reproducibility and analytical reliability, three consecutive spectra (triplicates) were acquired for each oil sample. Each spectrum was acquired with a total of 300 scans to obtain a good signal-to-noise ratio. The parameters used are described in Tables 6 and 7.

The raw mass spectra obtained in the FT-ICR MS were recalibrated internally using DataAnalysis software (Bruker, Billerica, Massachusetts, USA) using a known homologous series of oil constituents. From this recalibrated spectrum, the m/z and absolute intensity values for all peaks were obtained and exported in .asc files. For each sample analyzed, three subsequent injections were performed to obtain spectra in triplicate.

In petroleomics, data processing consists of several steps. The first step involves recalibrating the spectra in DataAnalysis for the subsequent assignment of molecular formulas by Composer 64 Version 1.5.3 software (Sierra Analytics Inc, Modesto, CA, USA). Both the recalibration of the spectra and the assignment of formulas were performed individually for each of the acquisitions.

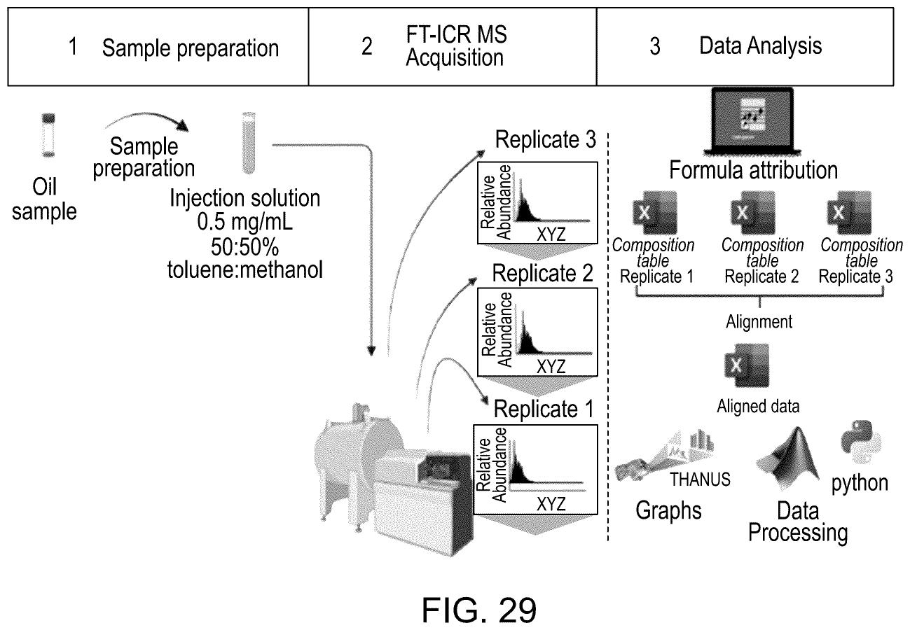

From the results of three spreadsheets called Composition Table, these were then used as input in a data alignment software (developed at LaCEM) to obtain a single data spreadsheet. Other programs can be used for data alignment to transform three spreadsheets into one. The process behind this alignment aims at maximizing the chemical information of each sample, through the combined evaluation of the replicates. FIG. 29 represents the flowchart comprising the steps involved in generating data used in Petroleomics, from sample preparation, spectra acquisition, assignment of molecular formulas, alignment of the triplicates, creation of graphs and, finally, more elaborate processing through data processing software. In other words, FIG. 29 illustrates all the steps involved from data preparation and acquisition, to data processing and generation of results and insights.

Several traditional petroleomics graphical tools available in the Thanus software, developed at LaCEM-UFG, were used to visualize and interpret the FT-ICR MS data. Several data analysis packages, routines, and algorithms developed in the laboratory for the Matlab 2020a (MathWorks Inc, Natick, Massachusetts, USA) and Python software were used for multivariate analysis. In this step, the aligned data spreadsheet resulting from the combination of the triplicates was used to obtain information regarding the class present in the samples, molecular formula, DBE, carbon number, monoisotopic abundance, and m/z for each of the samples to be analyzed.

From the data set formed for both APPI (+) and ESI (−), a traditional petroleomics analysis was performed, followed by an exploratory analysis and application of machine learning methods to build classification models, in addition to the selection of variables. These methods were applied to deepen the understanding of the molecular composition of the oil, in order to understand the influence of the type of operation for sample collection, as well as the geological formations.

Consequently, the use of these advanced strategies proved crucial to decipher the connectivity between the oil well fluids. Understanding the fluid interconnections is imperative not only for the reservoir characterization, but also has direct implications for the production efficiency. Knowledge about the connectivity influences decision-making regarding the reservoir management and maximization of the oil extraction, contributing to the increased productivity and reduced operating costs, aligning the operations with the inherent complexities of the reservoir.

### Analytical Reliability

From FT-ICR MS spectra, it is possible to obtain information regarding carbon and DBE distribution, class distribution, ternary graphs, among others, going beyond the identification of molecular components. In this way, the mass spectrometry becomes an ally of geochemists in evaluating the reservoir connectivity. However, the careful selection of the classes to be analyzed plays a crucial role in this process. In addition, the rigorous evaluation of the accuracy of the measurements is essential to ensure the reliability of the obtained results.

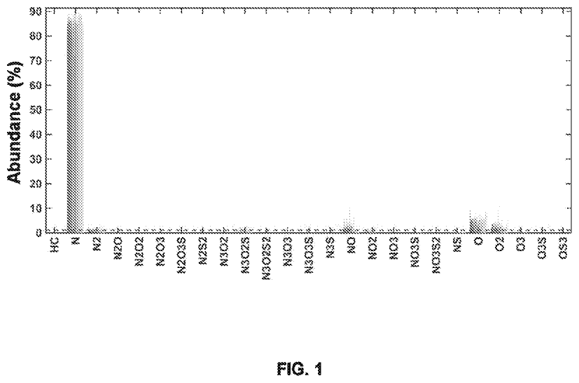

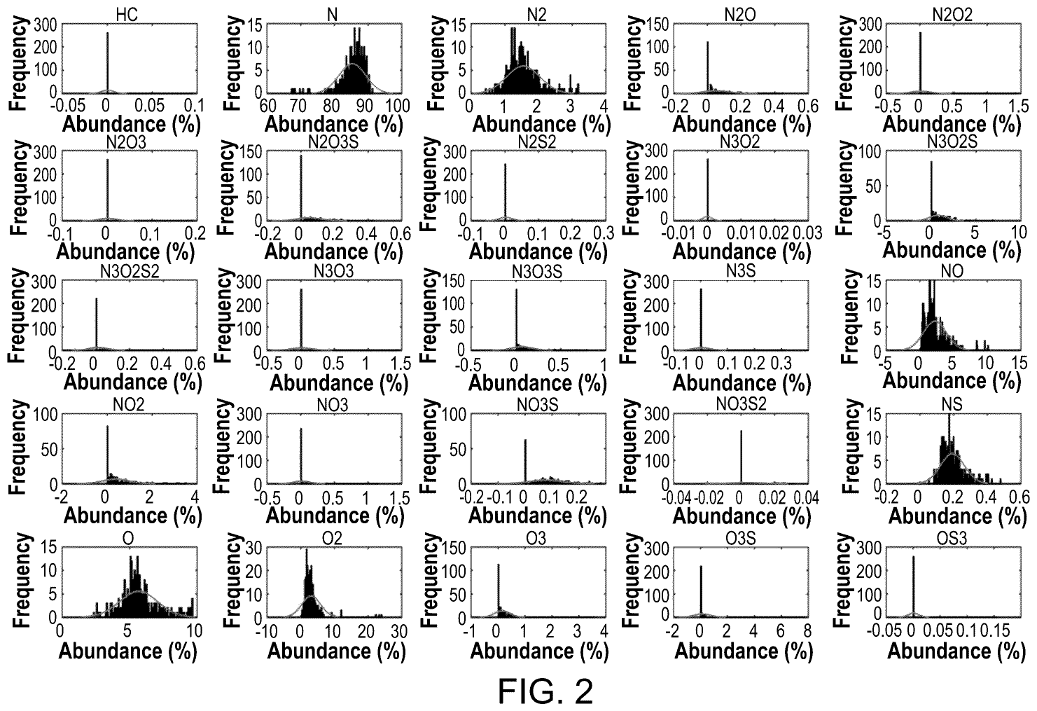

For ESI (−) analyses, the class distribution graphs, followed by histograms of frequency of occurrence of the classes are presented in FIGS. 1 and 2, respectively. It is possible to note that the classes N, N₂, NO, O and O₂ stand out not only for their abundance above 18, but also for their high frequency in a significant number of samples. This finding points to the predominance of these classes in the analyzed data set and justifies their selection for more in-depth analytical analyses.

In summary, the selection of the classes N, N₂, NO, O and O₂ for subsequent analyses is based on both their significant presence in terms of abundance and their high frequency of occurrence in the samples examined. Such a targeted focus allows for a detailed examination of the most impactful classes, enabling the identification of patterns or correlations relevant to the overall research objectives.

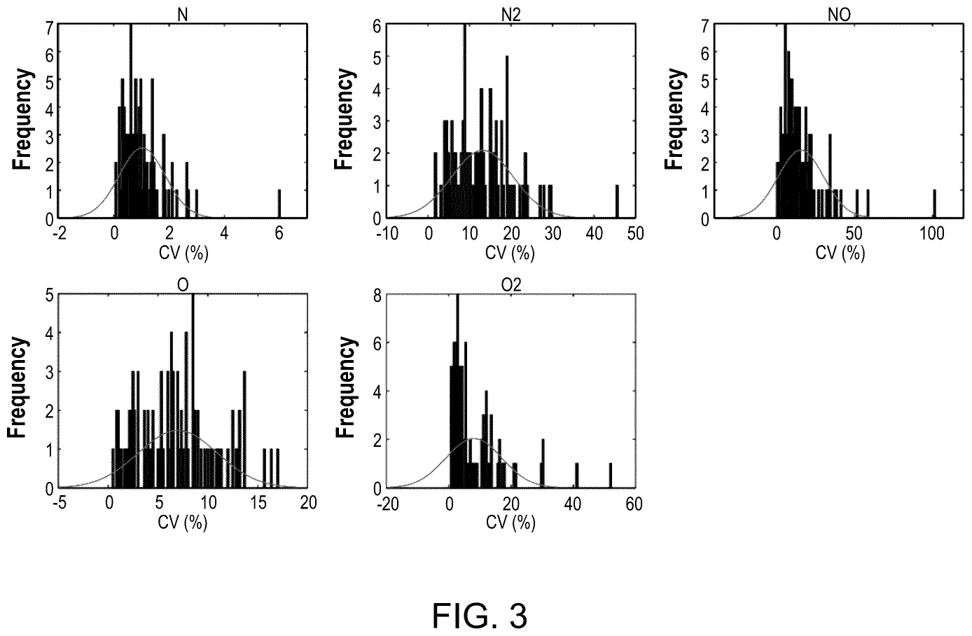

FIG. 3 provides a visual representation of the intrinsic variation of the classes N, N₂, NO, O and O₂ in the triplicates of the 88 samples, through the calculation of the coefficient of variation (CV). This statistical parameter is crucial to understand the dispersion of the data in relation to the mean.

The CV analysis allows the evaluation of the consistency and reliability of the measurements for each class. A low CV indicates less dispersion of data and, consequently, greater precision and reproducibility of the measurements. On the other hand, a high CV may indicate greater variability in the samples, which may be attributed to several factors, such as sample heterogeneity or technical inaccuracies.

Classes N and O stand out not only for their abundance, but also for the remarkable consistency evidenced by their low CVs. The greater presence of these classes in the samples may be an indication that their detection and measurement are more reliable and less susceptible to fluctuations, which is corroborated by the lower variation observed in the results. This stability may also reflect a lower sensitivity of these classes to variations in the analytical procedure or in the sample matrix, reinforcing their selection as robust indicators for more in-depth analyses.

In contrast, the classes N₂, NO and O₂, despite presenting relatively higher CVs, continue to be of interest due to their lower abundances. The more significant variations observed in these classes can be partially attributed to their lower relative abundance in the samples. Due to their lower representation, any small variation in the measurement process or in the sample composition can result in a larger percentage fluctuation in the CV, a well-known phenomenon in chemical and biochemical analysis.

The interpretation of the higher CVs for classes N₂, NO and O₂ must therefore be considered in the context of their lower abundance. These variations, although larger than those observed for classes N and O, remain within an acceptable range for complex analyses, where a certain tolerance for variability is understood and expected. The recognition of these dynamics is crucial for the interpretive integrity of the data and for making informed decisions about the future direction of investigations.

In summary, the higher abundance of classes N and O and the lower variation in their CVs qualify the same as excellent markers for continued analyses. Classes N₂, NO and O₂, despite their greater variations, are equally important and acceptable for subsequent studies, as long as these variations are contextualized within the complexity and lower abundance of these classes in the samples.

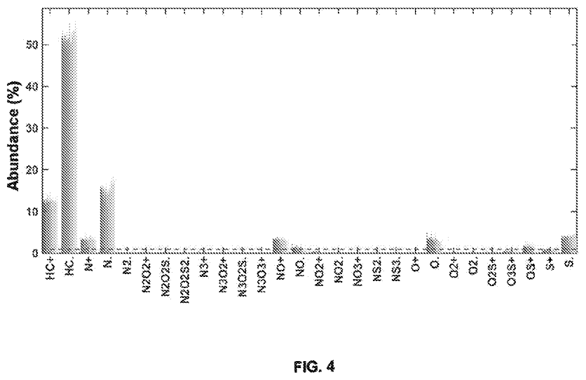

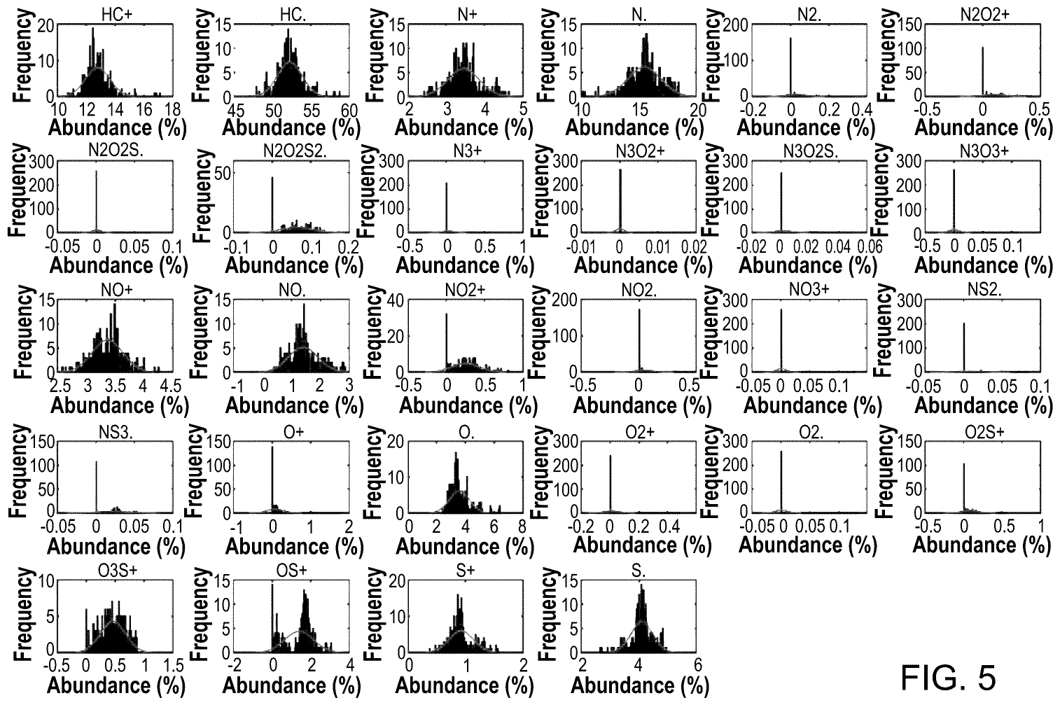

For spectra acquired by APPI (+), FIGS. 4 and 5 provide a detailed and quantitative view of the class distribution in a set of 88 samples analyzed in triplicate, allowing a careful evaluation of the relative abundance and consistency of the chemical classes studied.

The analysis of FIG. 4 reveals that some classes, such as HC, N, NO, O, OS and S, exhibit abundance peaks greater than 1% in most samples, suggesting consistency and importance in this set. Regarding FIG. 5, these classes show a high frequency of non-zero abundance values, highlighting their importance and consistency in the samples.

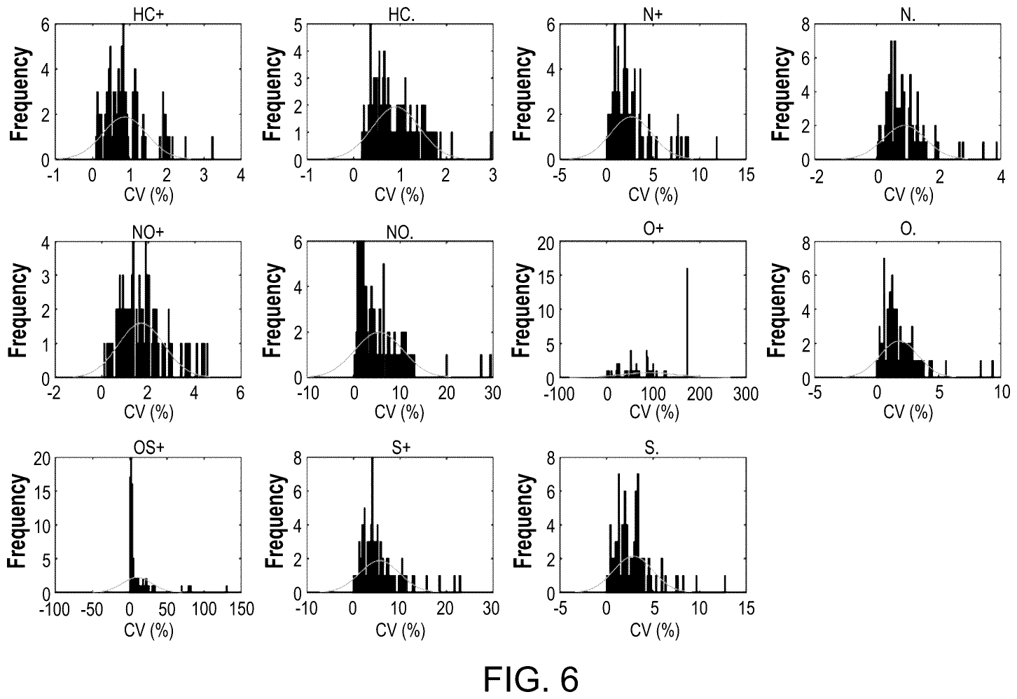

The classes HC⁺ and HC., N⁺ and N·, NO⁺ and NO., O⁺ and O·, OS⁺ and S⁺ and S· were identified as the most significant for future analyses due to their abundance and consistent presence in the samples. The choice to focus on these classes, considering both their radical and protonated forms, is grounded on the data presented and will be crucial for a detailed understanding of the underlying chemical properties and behaviors. Following the selection of the classes, FIG. 6 presents the analysis of the variability of these classes through the CV of each sample.

Through FIG. 6, it is possible to observe that the HC⁺ and HC· classes exhibit a relatively low CV, denoting a lower variability and a higher precision in the measurements. This consistency is essential to ensure the reliability of the data, especially in analyses that depend on the sensitive detection of chemical variations. The N⁺ and N. classes show a greater variation in the CV. However, most of the samples concentrate on lower CV values, which still indicates good stability for these classes. This suggests that the observed differences may be inherent to the properties of the samples or result from specific chemical processes.

For the NO⁺ and NO classes, a similar trend is observed with an acceptable variation in the CV, indicating that, despite the variations, the measurements are reliable for most of the samples. The O⁺ and O classes, despite exhibiting some extreme CV values, suggesting notable variations between the samples, generally maintain a consistency that justifies their selection for future analyses. Finally, the OS⁺ and S·, and S⁺ and S classes, present a variable behavior, with the S⁺ class showing a greater dispersion in the CV values. This observation can be attributed to the sensitivity of these classes to specific factors of the sampling environment or to the particularities of the analytical process.

Therefore, the CV analysis reinforces the choice of the HC, N, NO, O, OS and S classes for further analyses, with special attention to the variations observed in the protonated and radical forms. The variations in CV, although greater for some classes, remain within an acceptable spectrum, given the complexity of the samples and the analytical methods involved. This understanding of the variability is crucial for the adequate interpretation of the data and for the continuity of the investigations, ensuring that the conclusions are based on accurate and representative measurements of the samples.

After careful selection of the classes to be analyzed and careful evaluation of the accuracy of the measurements, it became possible to use the results in the evaluation of the compositional differences between samples from different types of operation (PVT and DST) and between samples from different types of geological formation and, finally, in the evaluation of the connectivity between the fluids in the reservoir wells.

### Operation Types

There was applied the method to differentiate oils from two types of operations: PVT and DST. The samples obtained by PVT operation undergo stirring and heating at 40° C. for homogenization before the analysis, while the samples obtained by DST do not undergo this process. In addition, some PVT samples were contaminated by drilling fluids during the sampling process and will be treated herein as another sample group (PVT-C).

Initially, the carbon number distribution and DBE distribution in the samples were evaluated. For ESI (−) analyses, the carbon number and DBE distributions of compounds of the classes N, N₂, NO, O and O₂ were evaluated. Meanwhile, for APPI (+) analyses, the evaluation was directed to the distribution of carbons and DBE of the compounds of the HC, N, NO, O, OS and S classes.

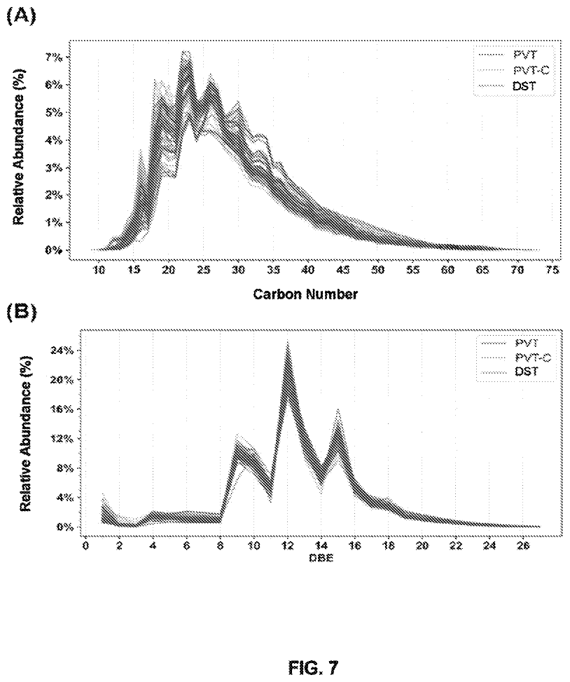

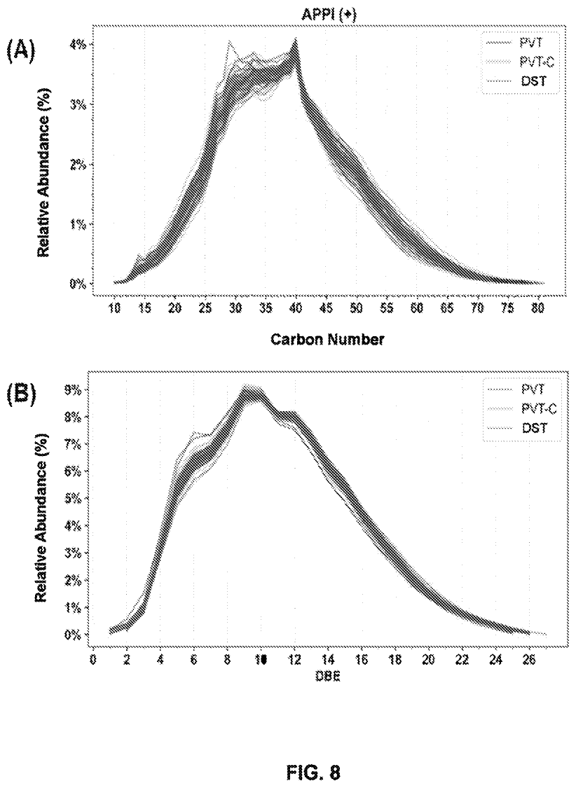

In FIG. 7A, it is possible to note that the compounds detected by ESI (−) in DST samples present a higher relative abundance of compounds with a higher number of carbons compared to the PVT and PVT-C samples. On the other hand, in FIG. 7B, it is noted that the PVT samples reach higher abundances of compounds with DBE 15, while the PVT-C samples stand out for presenting higher abundances of species with low DBE (<3), as well as for species with DBE 9 and 12. Meanwhile, species with DBE≥19 were detected in greater abundance in the DST samples. Additionally, graphs relating to the distribution of carbons and DBE for compounds detected by APPI (+) are presented in FIG. 8. In these, despite the smaller apparent variation, the DST samples also include species with higher carbon numbers and DBE.

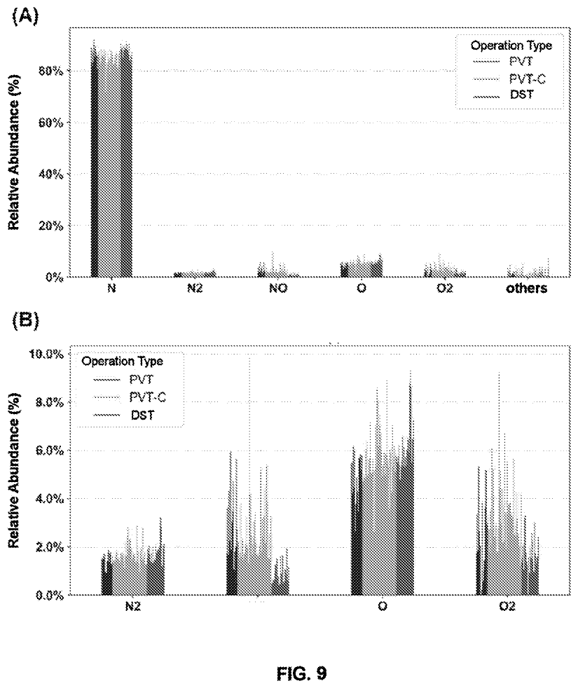

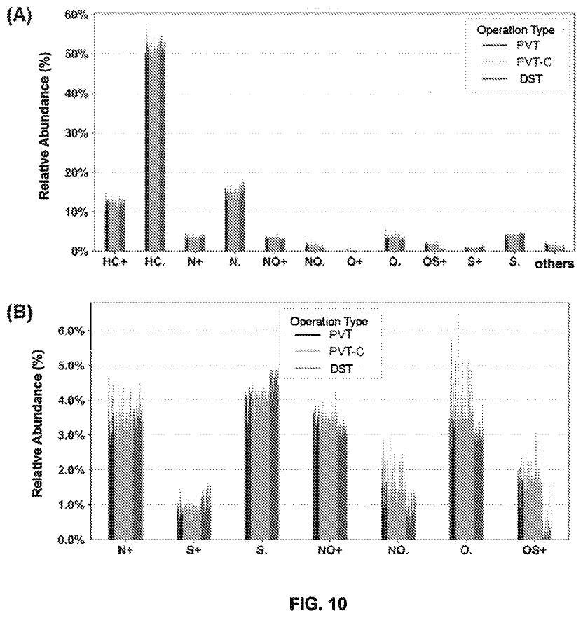

In addition to what was discussed, it is noted that, for both sources, the PVT and PVT-C samples present a greater apparent variation in the abundances, both in carbon numbers and DBE. After analyzing the distribution of carbons and DBE, the distribution of classes in the samples analyzed by ESI (−) and APPI (+) was evaluated, and the results are presented in FIGS. 9 and 10, respectively. For ESI (−), there is noted a little difference between the PVT and PVT-C samples, as for the differences between the PVT and DST samples, the greatest differences are observed for the classes containing O heteroatoms. Note that the NO and O₂ classes are more abundant in PVT samples, but the O class is detected in greater abundance in the DST samples.

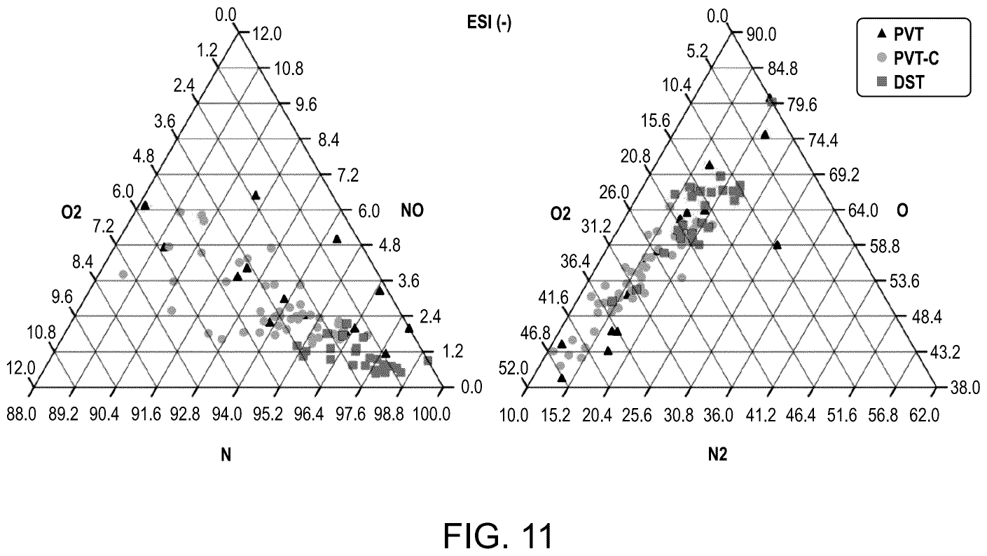

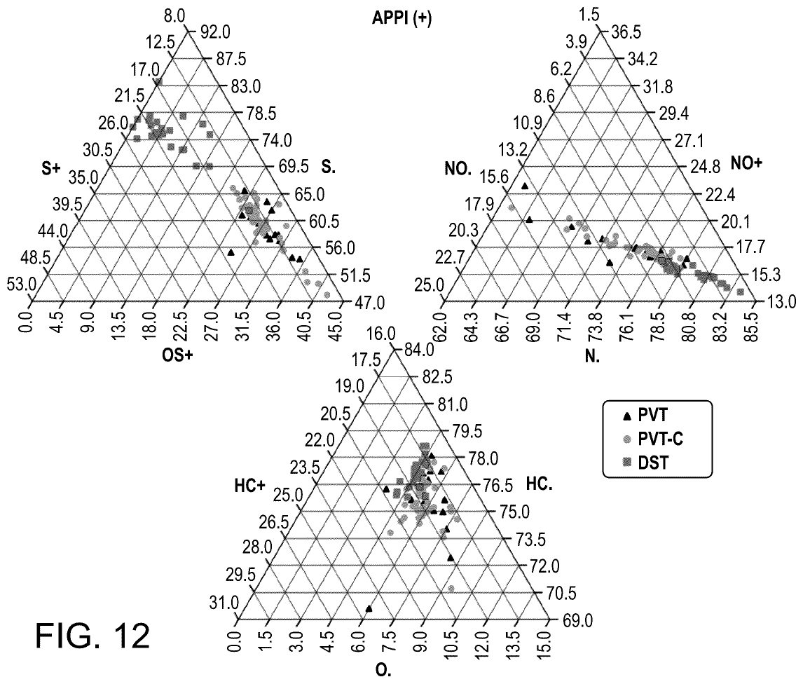

Similar results were obtained when the APPI (+) source was used. In FIG. 10, it can be noted that the PVT and PVT-C samples consistently present more classes containing oxygen. In addition, no significant differences are evidenced between the PVT and PVT-C samples. These conclusions are evidenced in the ternary graphs presented in FIGS. 11 and 12.

In FIG. 11, it is possible to notice a clear separation between the DST samples and the other sample groups: the DST samples present a greater amount of N and N₂ while the PVT and PVT-C samples present more NO, O and O₂. Similarly, in FIG. 12, the DST samples present a greater amount of S⁺, N·, HC· and HC⁺, while the PVT and PVT-C samples present more OS⁺, OS·, NO·, NO⁺ and O.

Due to this trend, it is believed that this compositional difference is caused by oxidative reactive processes that occur in the PVT and PVT-C samples during heating. This hypothesis reinforces the hypothesis of the compositional difference observed in the carbon number and DBE distributions, explored previously.

To corroborate this hypothesis, there was performed a theoretical analysis by molecular modeling using electronic structure calculations. In this case, a representative molecule was subjected to an oxidative process by an O₂ molecule at 40° C. For the model reaction, it was chosen to use a nucleus common to the hydrocarbons present in oils, indene, with molecular formula C₉H₈ (Stauffer et al., 2008). In addition, as will be discussed in the subsection Machine Learning of the Operation Type, indene was more frequent in the DST samples than in the PVT samples.

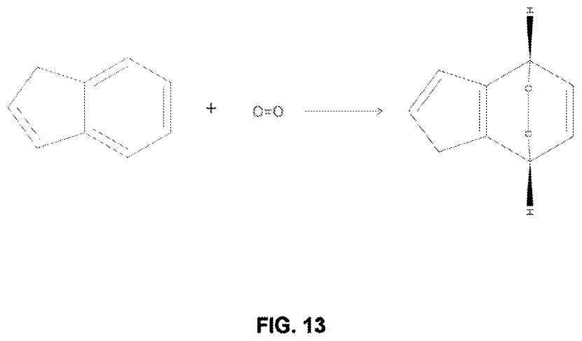

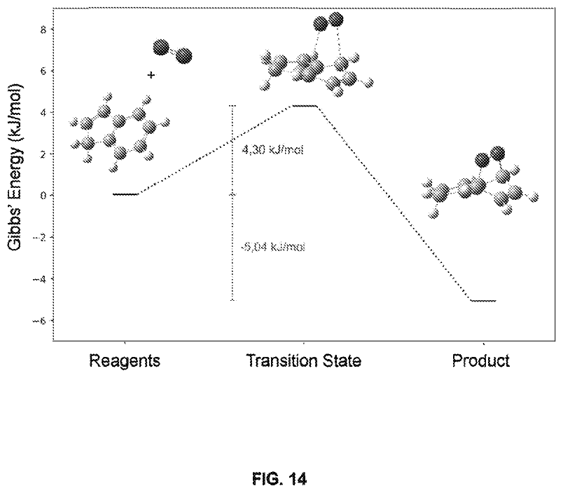

In the model reaction, the indene molecule is attacked by the O₂ molecule, forming an epoxide as a reaction product (FIG. 13). To illustrate this process, the potential energy surface of the reaction was constructed based on the Gibbs' energies calculated for the species involved, as shown in FIG. 14.

The potential energy surface shows a spontaneous reaction, where the Gibbs' free energy variation is-5.04 KJ/mol. This reaction presents a relatively low energy barrier of 2.30 KJ/mol, which contributes to a high reaction kinetic constant, recorded at 2×10⁻¹⁹ cm³ mol⁻¹ s⁻¹ at 40° C.

The spontaneity of the reaction and its fast rate under the temperature conditions applied to the model reaction suggest that the heating procedure used in the PVT operation probably plays a significant role in the compositional modification of the samples under study.

### Exploratory Analysis

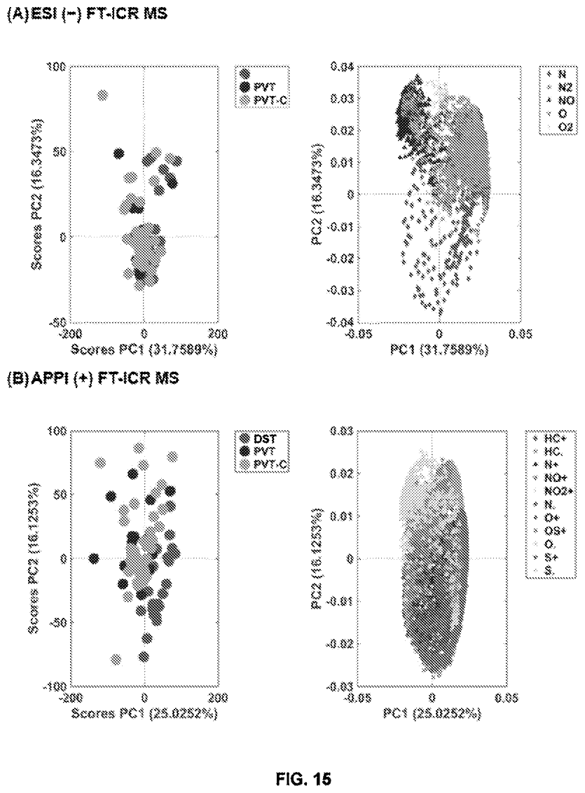

The principal component analysis (PCA) is a fundamental method in the processing and interpretation of high-dimensional data sets, such as those generated by advanced mass spectrometry techniques. In the type-of-operation study, there was evaluated the applicability of PCA on ESI (−) and APPI (+) FT-ICR MS data from 88 oil samples (FIG. 15).

PCA was applied to transform the original set of correlated variables into a new set of uncorrelated variables, the principal components, which are ordered so that the first component retains the greatest possible variation of the data, and each subsequent component, while orthogonal to the previous one, retains the maximum remaining variation.

In the ESI score graph (−), it can be seen that the first principal component (PC1) is responsible for 31.7589% of the variance in the data, suggesting that it captures the main differences between the oil samples. The second principal component (PC2), represented on the vertical axis, explains an additional percentage of 16.3473% of the variance, which indicates that it encompasses variations that PC1 does not capture. The distribution of the scores reveals a trend for the samples to group into specific classes, such as DST, PVT and PVT-C, each occupying different regions of the graph. However, although there is a grouping trend that suggests differences in the composition or properties of the oils, it is important to highlight the occurrence of overlap between these classes. This implies that, despite the general trends, there are areas where the classes are not perfectly discriminated by the PCA, suggesting similarities between them or the need for more components for a clearer separation.

Loadings in PCA refer to the variables or molecular formulas identified in the mass spectra. These points are distributed in relation to the axes of the first and second principal components (PC1 and PC2), and their location reflects how significant the contribution of each variable is to the total variation captured by these components. Variables that are far from the origin of the graph have a greater impact on the principal components, with those positioned extremely far to the right or left exerting a substantial weight on PC1, for example.

The spatial relation between loadings and scores is essential to decipher the underlying chemical composition of the oil samples. The loadings of the variables that align in the same direction as the scores of a specific class tend to be more characteristic of that class, suggesting a distinctive chemical profile. The presence of a dense overlap of loadings indicates that many variables affect the differentiation between the oil classes, pointing to a complexity in the data that may require a closer examination for a full understanding of the chemical nuances present.

Regarding APPI (+), in the scores graph, the first principal component (PC1) captures 25.0252% of the variance, a value that highlights the presence of significant variation, although smaller than that observed in the ESI analysis. The second component (PC2) contributes 16.1253% of the variation, indicating a similar distribution of variation between the two principal components compared to the ESI. The oil classes represented, DST, PVT and PVT-C, demonstrate a certain degree of dispersion in the PCA space, but the same substantial overlap between them as in the ESI.

Observing the loadings graph, the distribution of the variables does not show a clear trend of separation, suggesting that the chemical distinctions between the samples are subtle and not easily unraveled by this analysis. The density and mixing of variable classes along the PCA axes are a reflection of the diverse and complex chemical composition of the oils, and the correlation between the loadings and the oil classes is not immediately apparent, making the direct interpretation challenging.

The overall analysis of the scores, for both ESI and APPI, reveals a remarkable proximity between the PVT and PVT-C classes, suggesting a substantial similarity in their chemical compositions. This observation is consistent across both ionization techniques, indicating that the differences between PVT and PVT-C may be smaller than previously realized by the radar graphs. Given this evidence, for the subsequent analyses, it was decided to consolidate PVT and PVT-C into a single category, called PVT. This consolidation is essential to advance the interpretation of the data and to ensure accuracy in the predictive modeling and discriminant analysis steps that will follow.

As observed in the results obtained by PCA, this method served as a valuable preliminary exploratory tool in the investigation herein of the different types of operation. Although it allowed a general visualization and a certain degree of discrimination between the classes, the significant overlaps between the same suggest the need for more refined analytical methods for a detailed characterization.

In this context, an advance was made towards the application of machine learning methods that are suitable for treating complex and correlated multivariate data such as the one herein. This type of approach will not only facilitate the distinction between the oil operation classes, but will also allow the identification of the variables that are most influential for this separation.

### Machine Learning

This section details the results obtained by using partial least squares regression for discriminant analysis (PLS-DA) with the method of selection of variables called ordered predictor selection for discriminant analysis (OPSDA). The PLS-DA approach is recognized for its ability to deal with multicollinearity in data, and the OPSDA complements this method by improving the selection of variables, ensuring that only the most relevant predictors are included. This combination aims at maximizing the discrimination between the PVT and DST samples, allowing the extraction of the subtle differences and intrinsic similarities. The modeling was restricted to these two categories of samples in order to generate more accurate and focused insights, allowing a deeper understanding of the characteristics that differentiate the PVT and DST processes. The results of the machine learning models presented below offer a new perspective on the data, enhancing decision-making and the evidence-based exploration strategy.

The PLS-DA OPSDA models were built to separate the PVT (64 samples) and DST (24 samples) classes using the data obtained by ESI (−) and APPI (+) FT-ICR MS. Five models were built for the ESI (−) FT-ICR MS data, corresponding to the N, N₂, NO, O, and O₂ variable classes selected for data processing. For APPI (+) FT-ICR MS, 11 different classes were selected between protonated and radical, namely: HC⁺, HC·, N⁺, N·, NO⁺, NO·, O⁺, O·, S⁺, S·, and OS⁺. To build the models in APPI (+), each class of variables was considered only once, considering the protonated and radical variables at the same time. In this way, for APPI (+), six models were built: HC, N, NO, O, S, and OS. Table 1 presents the sensitivity and accuracy of the classification models obtained for the PLS-DA OPSDA models to separate the type of operation by APPI (+) and ESI (−).

TABLE 1
Sensitivity and accuracy of the PLS-DA OPSDA models
regarding the type of operation for each class of
variables obtained by ESI (−) and APPI (+) FT-ICR MS.

| ESI (−) | Sensitivity | Accuracy | ESI (−) | Sensitivity | Accuracy |
|---|---|---|---|---|---|
| N | (%) | (%) | N₂ | (%) | (%) |
| DST | 100 | 100 | DST | 100 | 100 |
| PVT | 100 | 100 | PVT | 100 | 100 |
| ESI (−) | Sensitivity | Accuracy | ESI (−) | Sensitivity | Accuracy |
| NO | (%) | (%) | O | (%) | (%) |
| DST | 88 | 88 | DST | 96 | 93 |
| PVT | 88 | 88 | PVT | 92 | 93 |

| ESI (−) | Sensitivity | Accuracy | APPI (+) | Sensitivity | Accuracy |
|---|---|---|---|---|---|
| O₂ | (%) | (%) | HC | (%) | (%) |
| DST | 79 | 88 | DST | 100 | 100 |
| PVT | 91 | 88 | PVT | 100 | 100 |

| APPI (+) | Sensitivity | Accuracy | APPI (+) | Sensitivity | Accuracy |
|---|---|---|---|---|---|
| N | (%) | (%) | NO | (%) | (%) |
| DST | 100 | 100 | DST | 100 | 100 |
| PVT | 100 | 100 | PVT | 100 | 100 |
| APPI (+) | Sensitivity | Accuracy | APPI (+) | Sensitivity | Accuracy |
| O | (%) | (%) | S | (%) | (%) |
| DST | 100 | 99 | DST | 100 | 100 |
| PVT | 98 | 99 | PVT | 100 | 100 |

| APPI (+) | Sensitivity | Accuracy |
|---|---|---|
| OS | (%) | (%) |
| DST | 100 | 100 |
| PVT | 100 | 100 |

Table 1 illustrates the performance of the PLS-DA models, using sensitivity and accuracy as main metrics. These metrics validate the ability of the machine learning models to correctly classify samples, in which the sensitivity measures the ability of the model to identify true positives, while the accuracy reflects the proportion of correct predictions overall.

In the models using ESI (−), perfect sensitivity and accuracy (100%) are observed for DST and PVT samples in the N and N₂ categories, showing a clear and precise distinction of these samples. For ESI (−) NO, O, and O₂, the DST and PVT samples also demonstrated high levels of sensitivity and accuracy, although with a slight reduction compared to the N and N₂ categories.

Regarding the APPI (+) models, equally high sensitivity and accuracy are observed for DST and PVT samples in several categories, including HC, N, NO, and S, all reaching 100%. This indicates that the models are extremely effective in correctly identifying the samples for these classes of compounds.

The overall results highlight the robustness and accuracy of the PLS-DA OPSDA models in the context of petroleomics, confirming their usefulness in identifying and differentiating between the PVT and DST samples with high confidence.

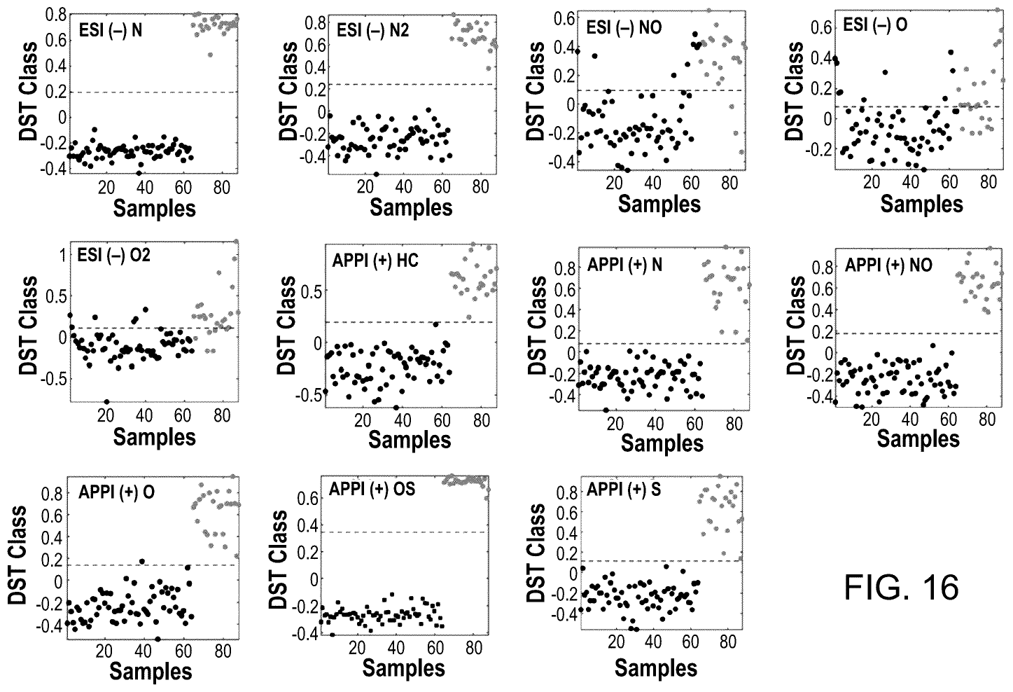

The classification results of the PVT samples are shown in FIG. 16 for the ESI (−) and APPI (+) FT-ICR MS models. Each graph illustrates the classification of the DST samples above the horizontal dotted line that represents the threshold for separation between the classes. Samples above this threshold are classified as belonging to the DST class, while samples below the line are classified as belonging to the PVT class.

In the context of this graph, the dotted line not only serves as a cutoff point for classification, but also highlights the clear distinction between the two classes. The DST samples, when all above the line, indicate a correct classification for this class, while the PVT samples, when all below the line, also indicate a correct classification. In this case, it is possible to have another graph where the opposite relation would be seen, with PVT samples above the threshold and PVT below. However, in the case of a classification problem with only two classes, only one graph is necessary to demonstrate the separation.

Therefore, the results in FIG. 16 are useful to visualize the degree of overlap or separation between the predicted classes and to evaluate the model performance in terms of sensitivity for each class. The absence of overlap is an indication that the model has a good discriminatory capacity for the analyzed classes. In summary, the results indicate that the PLS-DA model, with selection of variables by OPSDA, is highly effective in discriminating between DST and PVT samples for some classes of variables, both ESI (−) and APPI (+). These results emphasize the utility of machine learning models in contexts in which the classification accuracy is crucial, and highlight the importance of careful selection of variables to optimize the model performance.

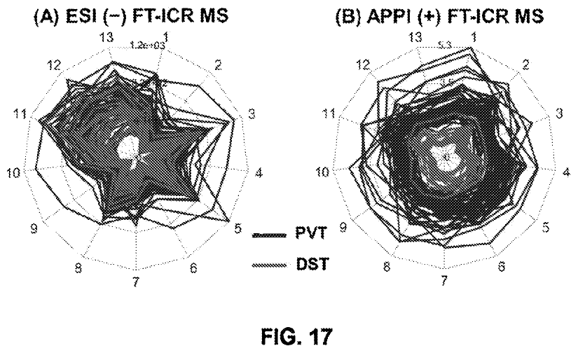

The most important variables for the classification of each of the 11 classification models with the ESI (−) and APPI (+) heteroatom classes, i.e., the molecular formulas, can be seen in Table 8 and Table 9, respectively. These variables, representing specific molecular formulas, were subjected to an optimization process, where the search for optimal ratios between pairs of variables was conducted. The objective of this refinement is to improve the distinction between the PVT and DST samples. Consequently, the ratios between the variables that presented the highest weights assigned in the optimization were highlighted in a radar graph (FIG. 17) and are presented in Table 2. This visual representation not only illustrates the separation of the two classes, but also provides insights into the relations between the ratios between variables that define the differences between the PVT and DST samples.

TABLE 2
Ratios of variables for optimized separation of DST and
PVT for ESI (−) and APPI (+) FT-ICR MS data.

| | ESI (−) FT-ICR MS | APPI (+) FT-ICR MS |
|---|---|---|
| 1 | C₁₉H₁₇N/C₁₈H₂₃N | C₃₀H₅₂OS/C₂₉H₅₆S |
| 2 | C₁₉H₁₇N/C₂₉H₄₅N | C₃₀H₅₂OS/C₆₇H₉₄ |
| 3 | C₁₉H₁₇N/C₄₁H₃₅N | C₃₀H₅₂OS/C₃₀H₅₈S |
| 4 | C₁₉H₁₇N/C₂₇H₄₁N | C₃₀H₅₂OS/C₆₉H₁₂₈ |
| 5 | C₁₉H₁₇N/C₃₅H₃₄N₂ | C₃₀H₅₂OS/C₆₈H₉₈ |
| 6 | C₁₉H₁₇N/C₃₀H₄₇N | C₃₀H₅₂OS/C₇₀H₁₂₈ |
| 7 | C₂₀H₁₉N/C₁₈H₂₃N | C₃₀H₅₂OS/C₅₅H₆₄ |
| 8 | C₁₉H₁₇N/C₄₀H₃₅N | C₃₀H₅₂OS/C₃₈H₃₁N |
| 9 | C₁₉H₁₇N/C₂₈H₄₃N | C₃₃H₅₈OS/C₂₉H₅₆S |
| 10 | C₁₉H₁₇N/C₄₂H₃₉N | C₃₀H₅₂OS/C₂₈H₅₄S |
| 11 | C₁₉H₁₇N/C₅₅H₆₉N | C₃₀H₅₂OS/C₆₈H₁₀₀ |
| 12 | C₁₉H₁₇N/C₃₈H₃₈N₂ | C₃₀H₅₂OS/C₃₉H₃₀ |
| 13 | C₁₉H₁₇N/C₃₃H₃₀N₂ | C₃₁H₅₆OS/C₂₉H₅₆S |

FIGS. 17A and 17B display all of the samples analyzed, highlighting the overall distinction between PVT and DST in all optimized ratios. The distribution of the samples in these graphs suggests a clear separation between the two sample classes, with the dispersion of the data allowing the identification of distinct patterns between the PVT and DST operation types. In turn, FIGS. 17C and 17D focus on a determined module, Module 5, displaying a subset of the samples to demonstrate that the differentiation observed in the total set is maintained at a modular level. This consistency in the separation between PVT and DST within Module 5 is representative of what is observed in the other modules.

### Geological Formation

The 88 samples investigated originate from four distinct geological formations (A, B, C and D). In this section, the characterization data from ESI (−) and APPI (+) FT-ICR MS analyses will be used to discern the samples based on their geological formation. Initially, the carbon number distribution and the DBE distribution in the samples were examined. In the ESI (−) analyses, the carbon number and DBE distributions for compounds of the classes N, N₂, NO, O and O₂ were investigated. At the same time, in the APPI (+) analyses, the analysis focused on the distribution of carbons and DBE for compounds of the HC, N, NO, O, OS and S classes.

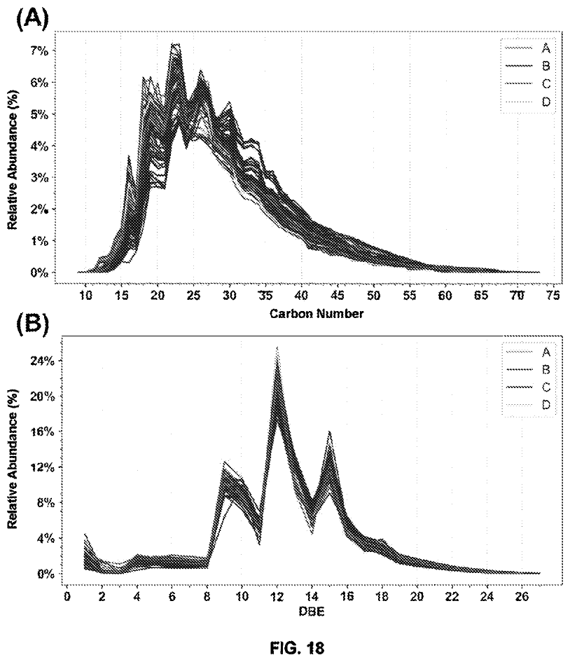

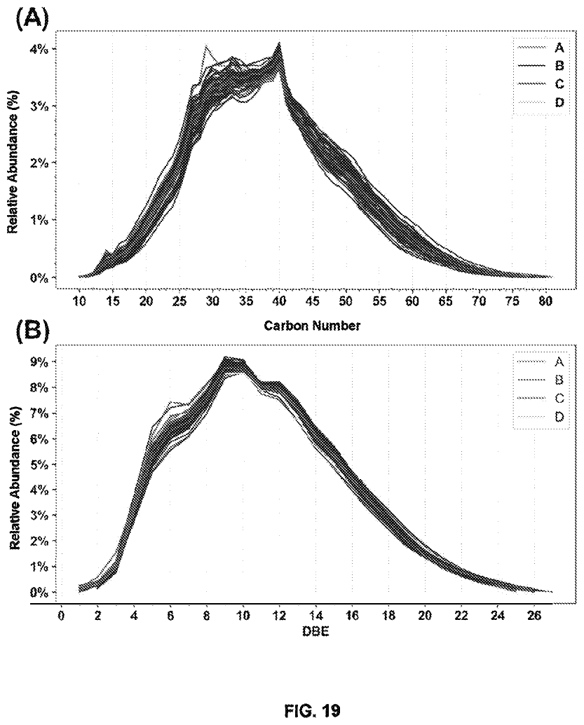

In FIG. 18A, albeit subtly, it is possible to observe that the compounds identified by ESI (−) in samples from formation B present a greater relative abundance of compounds with a greater number of carbons (≥23) compared to samples from other formations. On the other hand, in FIG. 18B, a smaller apparent variation is noted between the DBE distributions for samples from different geological formations. Additionally, the graphs relating to the distribution of carbons and DBE for compounds identified by APPI (+) are presented in FIG. 19. In the same, despite the smaller apparent variation, the samples from formation B also include species with higher numbers of carbons. Regarding the DBE distribution, no significant trends are observed that differentiate the samples according to the geological formation.

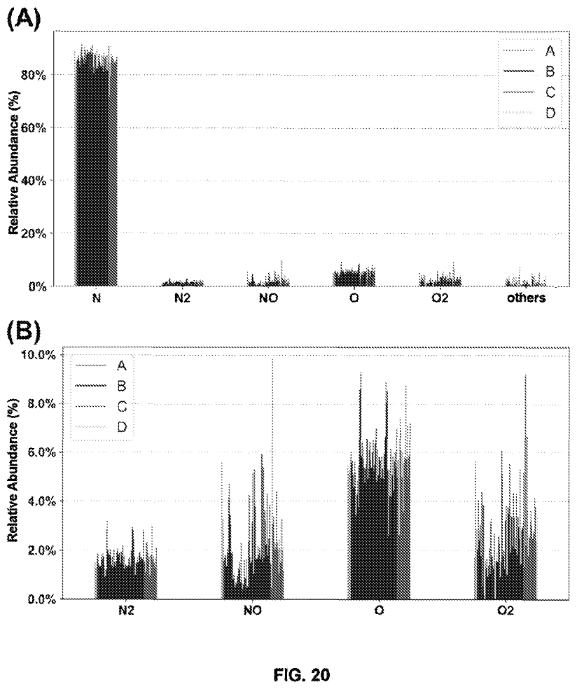

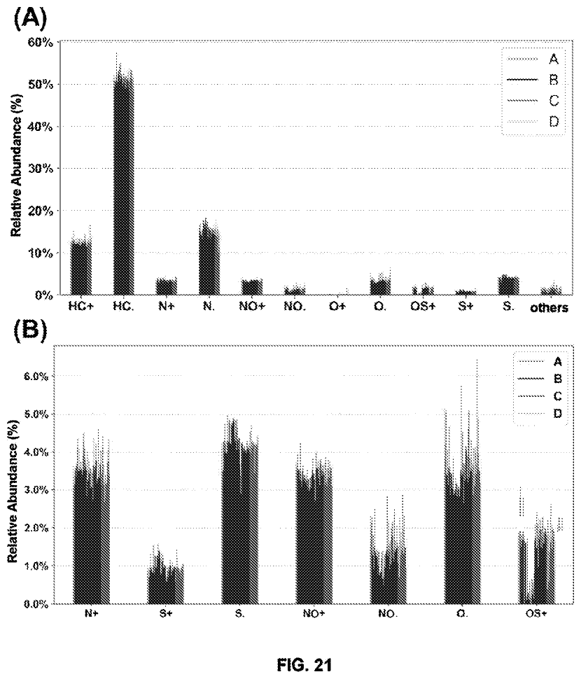

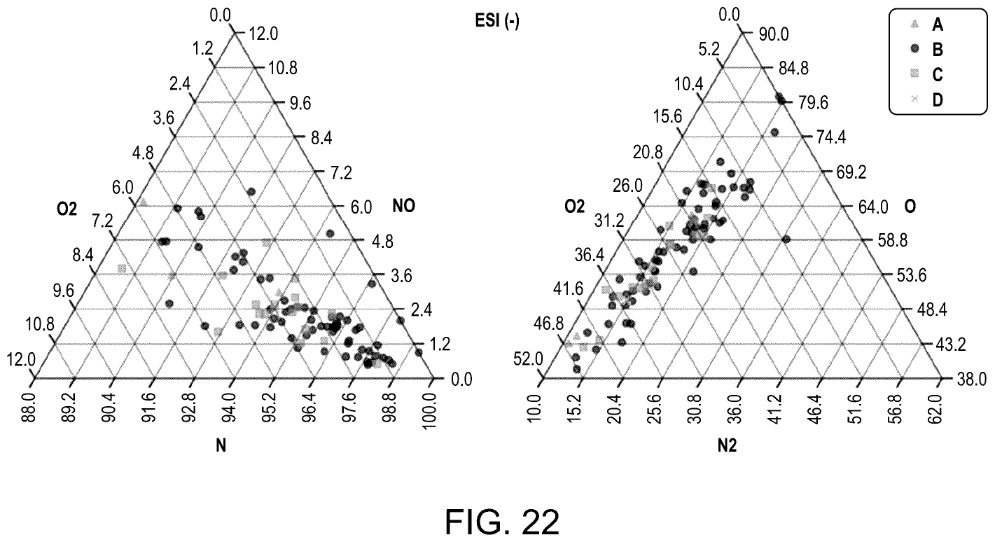

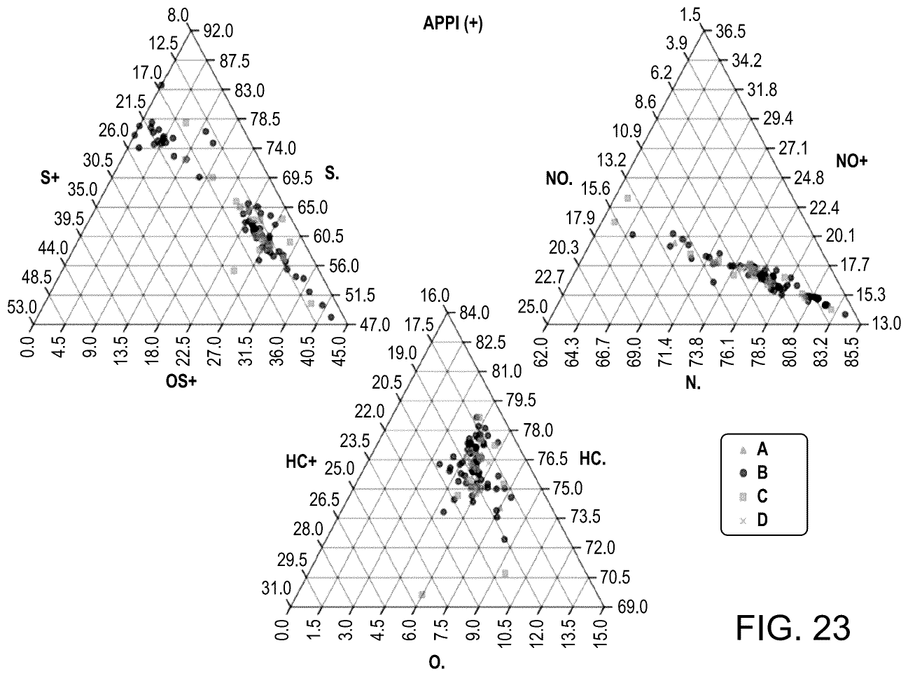

FIGS. 20 and 21 show the class distributions for the samples analyzed by ESI (−) and APPI (+) FT-ICR MS. However, for the results obtained by both ionization sources, it was not possible to observe a clear trend that would allow the samples to be differentiated according to their geological formation. Based on this, it can be inferred that, regardless of the geological formation, the oils present compositional similarity. This result can be interpreted as indicative of a good connectivity between the formations within the reservoir. These conclusions are evidenced in the ternary graphs presented in FIGS. 22 and 23.

The uniformity observed in the samples indicates a challenge in the characterization of the oil that transcends the capacity of traditional petroleomics. To overcome this complexity and unravel the nuances in the compositional differences between the samples from the diverse geological formations, the use of advanced statistical methods was made. This approach allowed for a deeper investigation of the distinctive characteristics of the samples, enabling a broader understanding of the subtle variations. In the following analysis steps, the focus was exclusively on the samples from formations B and C, due to the quantitative limitation of the samples from the other formations, which had only 5 samples. Such a selective criterion aimed at ensuring the robustness and the statistical validity of the obtained insights, focusing on the formations with a more representative data set.

### Exploratory Analysis

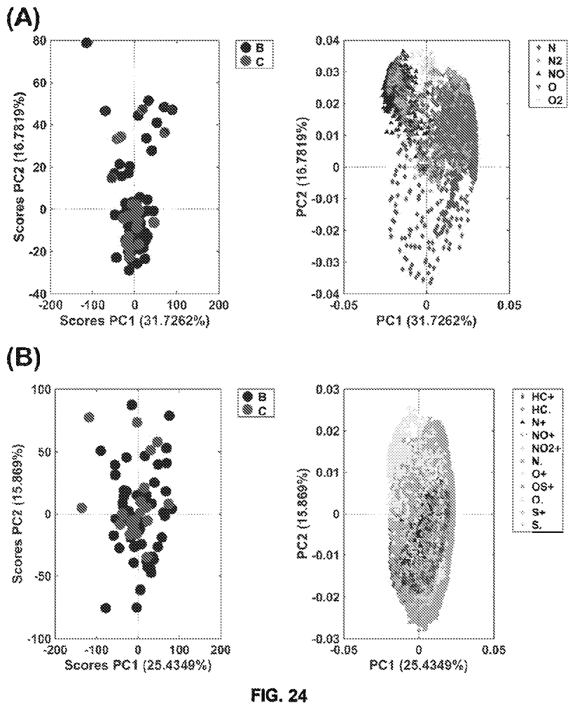

In the study of the geological formation, the applicability of PCA was evaluated on the ESI (−) and APPI (+) FT-ICR MS data of 83 oil samples (FIG. 24).

In the ESI (−) score graph, it was noted that the first principal component (PC1) is responsible for 31.7263% of the variance in the data, suggesting that it captures the main differences between the oil samples. The second principal component (PC2), represented on the vertical axis, explains an additional 16.7819% of the variance, indicating that it encompasses variations that PC1 does not capture. The distribution of scores reveals that there is no trend for samples from the same formation to group together, suggesting similarities between them or the need for more components for a clearer separation.

Regarding APPI (+), in the score graph, the first principal component (PC1) captures 25.4349% of the variance, a value that highlights the presence of significant variation, although smaller than that observed in the ESI analysis. The second component (PC2) contributes 15.8690% of the variation, indicating a smaller distribution of variation between the two principal components compared to ESI. The oil geological formation classes represented are widely dispersed in the PCA space, indicating no trend for separation.

Based on the results obtained by PCA, it was concluded that this method served as a preliminary exploratory tool in the investigation of the geological formation. However, this analysis did not indicate a separation between samples from different geological formations, making it essential to apply more advanced statistical methods.

In this context, an advance was made towards the application of machine learning methods that are suitable for treating complex and correlated multivariate data such as those of the present invention. This type of approach will not only facilitate the distinction between the different geological formations of the oils, but will also allow the identification of the variables that are most influential for this separation.

### Machine Learning

In this section, the results obtained by using PLS-DA with the OPSDA method of selection of variables for analyzing formation types will be detailed. This combination aims at maximizing the discrimination between the samples from the formations B and C, which allows the extraction of subtle differences and intrinsic similarities. The results of the machine learning models presented below provide a new perspective on the data, enhancing decision-making and evidence-based exploration strategy.

The PLS-DA OPSDA models were built to separate the classes of the formation B (63 samples) and the formation C (25 samples) using the data obtained by ESI (−) and APPI (+) FT-ICR MS. Five models were built for the ESI (−) FT-ICR MS data, corresponding to the classes of variables N, No, NO, O and O₂ selected for data processing. For APPI (+) FT-ICR MS, 11 different classes were selected between protonated and radical, namely: HC⁺, HC·, N⁺, N·, NO⁺, NO·, O⁺, O·, S⁺, S·, and OS+. To construct the models in APPI (+), each class of variables was considered only once, considering protonated and radical variables at the same time. In this way, six models were built for APPI (+): HC, N, NO, O, S, and OS. Table 3 shows the sensitivity and accuracy of the classification models obtained for the PLS-DA OPSDA models to separate the type of operation by APPI (+) and ESI (−).

Table 3 illustrates the performance of the PLS-DA models, using sensitivity and accuracy as the main metrics. These metrics validate the ability of the machine learning models to correctly classify the samples: the sensitivity measures the ability of the model to identify the true positives, while accuracy reflects the proportion of correct predictions in general.

TABLE 3
Sensitivity and accuracy of the PLS-DA OPSDA models regarding
the geological formation for each class of variables
obtained by ESI (−) and APPI (+) FT-ICR MS.

| ESI (−) | Sensitivity | Accuracy | ESI (−) | Sensitivity | Accuracy |
|---|---|---|---|---|---|
| N | (%) | (%) | N₂ | (%) | (%) |
| Formation B | 94 | 85 | BV | 100 | 100 |
| Formation C | 60 | 85 | IT | 100 | 100 |
| ESI (−) | Sensitivity | Accuracy | ESI (−) | Sensitivity | Accuracy |
| NO | (%) | (%) | O | (%) | (%) |
| Formation B | 100 | 100 | BV | 92 | 90 |
| Formation C | 100 | 100 | IT | 85 | 90 |

| ESI (−) | Sensitivity | Accuracy | APPI (+) | Sensitivity | Accuracy |
|---|---|---|---|---|---|
| O₂ | (%) | (%) | HC | (%) | (%) |
| Formation B | 73 | 67 | BV | 94 | 89 |
| Formation C | 50 | 67 | IT | 75 | 89 |
| APPI (+) | Sensitivity | Accuracy | APPI (+) | Sensitivity | Accuracy |
| N | (%) | (%) | NO | (%) | (%) |
| Formation B | 100 | 100 | BV | 97 | 96 |
| Formation C | 100 | 100 | IT | 95 | 96 |
| APPI (+) | Sensitivity | Accuracy | APPI (+) | Sensitivity | Accuracy |
| O | (%) | (%) | S | (%) | (%) |
| Formation B | 76 | 67 | BV | 100 | 100 |
| Formation C | 40 | 67 | IT | 100 | 100 |

| APPI (+) | Sensitivity | Accuracy |
|---|---|---|
| OS | (%) | (%) |
| Formation B | 100 | 99 |
| Formation C | 95 | 99 |

In models using ESI (−), a sensitivity and perfect accuracy (100%) for formation B and formation C in the N₂ and NO categories, showing a clear and precise distinction of these samples. For ESI (−) N, O, and O₂, formation B and C presented lower levels of sensitivity and accuracy, mainly for the samples of formation C.

Regarding the APPI (+) models, equally high sensitivity and accuracy are observed for the samples of formation B and C for the N and S classes, both reaching 100%. This indicates that the models are extremely effective in correctly identifying samples from different formations for these classes of compounds.

The classification results of the samples of the formation B are shown in FIG. 24 for the ESI (−) and APPI (+) FT-ICR MS models. Each graph illustrates the classification of the samples of the formation B above the horizontal dotted line that represents the threshold of separation between the classes. Samples above this threshold are classified as belonging to the formation B, while samples below the line are classified as belonging to the formation C.

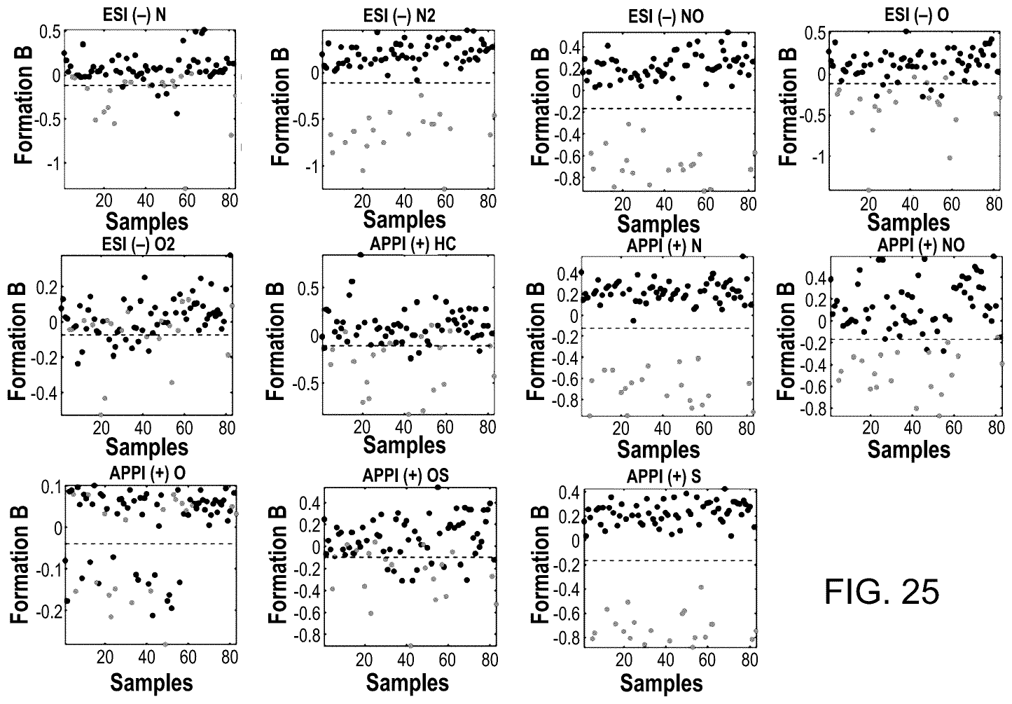

The results presented in FIG. 25 are useful to visualize the degree of overlap or separation between the predicted classes and to evaluate the model performance in terms of sensitivity for each class. The absence of overlap is an indication that the model has a good discriminatory capacity for the analyzed classes. In summary, the results indicate that the PLS-DA model, with the selection of variables by OPSDA, is highly effective in discriminating between samples from the formations B and C for some classes of variables, both ESI (−) and APPI (+). These results emphasize the usefulness of machine learning models in contexts in which the classification accuracy is crucial, and highlight the importance of a careful selection of variables to optimize the model performance.

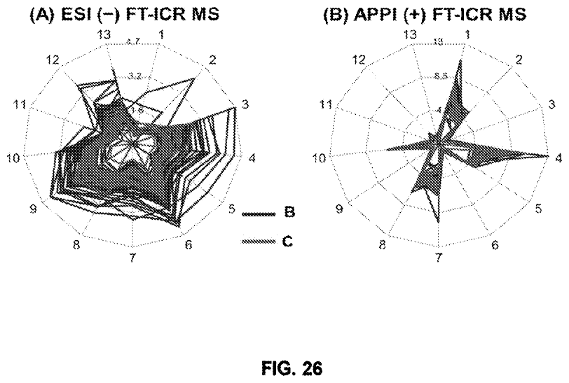

The most important variables for the classification of each of the 11 classification models with the ESI (−) and APPI (+) heteroatom classes, i.e., the molecular formulas, can be seen, respectively, in Tables 10 and 11. These variables, representing specific molecular formulas, were subjected to an optimization process, where the search for optimal ratios between pairs of variables was conducted. The objective of this refinement is to improve the distinction between the samples of the formations B and C. Consequently, the ratios between the variables that presented the highest weights assigned in the optimization were highlighted in a radar graph (FIG. 26) are presented in Table 4. This visual representation not only illustrates the separation of the two classes, but also offers insights into the relations between the ratios between variables that define the differences between the samples of the formations B and C.

TABLE 4
Ratios of variables for optimized separation of BV and
IT for ESI (−) and APPI (+) FT-ICR MS data.

| | ESI (−) FT-ICR MS | APPI (+) FT-ICR MS |
|---|---|---|
| 1 | C₂₃H₃₀O₂/C₂₁H₂₂N₂ | C₁₉H₃₂/C₅₉H₁₁₀ |
| 2 | C₂₃H₃₀O₂/C₃₄H₂₃N | C₁₉H₃₂/C₁₂H₁₈ |
| 3 | C₅₆H₉₁N/C₂₁H₂₂N₂ | C₄₁H₇₈S/C₅₉H₁₁₀ |
| 4 | C₅₇H₈₇N/C₂₁H₂₂N₂ | C₁₉H₃₀/C₅₉H₁₁₀ |
| 5 | C₅₈H₈₇N/C₂₁H₂₂N₂ | C₁₉H₃₂/C₂₀H₃₆ |
| 6 | C₅₇H₈₇N/C₃₄H₂₃N | C₄₀H₇₈S/C₅₉H₁₁₀ |
| 7 | C₅₆H₉₃N/C₂₁H₂₂N₂ | C₁₉H₃₂/C₅₉H₁₀₈ |
| 8 | C₅₈H₈₇N/C₃₄H₂₃N | C₁₉H₃₀/C₁₂H₁₈ |
| 9 | C₅₆H₉₁N/C₃₄H₂₃N | C₄₁H₇₈S/C₅₈H₁₀₈ |
| 10 | C₅₆H₉₃N/C₃₄H₂₃N | C₁₉H₃₀/C₂₀H₃₆ |
| 11 | C₆₂H₈₉N/C₃₄H₂₃N | C₄₀H₇₈S/C₅₈H₁₀₈ |
| 12 | C₅₆H₇₇N/C₂₁H₂₂N₂ | C₃₈H₇₂S/C₅₉H₁₁₀ |
| 13 | C₃₂H₄₄O/C₂₁H₂₂N₂ | C₄₁H₇₈S/C₁₉H₃₄ |

FIGS. 26A and 26B show the totality of the samples analyzed, highlighting the overall distinction between the B and C formations in all the optimized ratios. The distribution of the samples in ESI (−) suggests a clear separation between the two sample classes, with data dispersion allowing the identification of distinct patterns between the geological formations B and C.

### Connectivity of Reservoir Fluids

In this section, data generated by APPI (+) FT-ICR MS analyses were used to evaluate the vertical and lateral fluid connectivity, i.e., within the same well and between adjacent wells, respectively. For this purpose, the PCA method was used, in which the distance between scores was used to evaluate the differences and similarities between the analyzed samples. In this way, a greater proximity or overlap between scores is interpreted as a greater similarity between samples, and is therefore representative of the communication (or absence of barriers) between fluids.

In reservoir X, there are 88 samples divided into 24 wells and 11 modules, divided as follows:
Module 1: Wells 8, 11 and 25;
Module 2: Wells 3, 10, 15 and 17;
Module 3: Wells 14 and 24;
Module 4: Wells 9, 22 and 26;
Module 5: Wells 2, 7 and 20;
Module 6: Well 27;
Module 7: Well 5;
Module 8: Wells 6 and 19;
Module 9: Wells 13 and 16;
Module 10: Wells 1 and 12; and
Module 12: Well 21.

During the fluid connectivity analysis, certain methodological limitations influenced the approach in some modules. Specifically, Module 9, which comprises Wells 13 and 16, presented a significant limitation, since all its samples were collected using only the PVT method. The proposed connectivity analysis requires data from both collection types, PVT and DST, for a conclusive evaluation. In this way, the absence of DST samples in this module made the connectivity analysis inconclusive.

On the other hand, Modules 6 and 7, which include, respectively, Wells 27 and 5, have only one well each. This restricted the analysis herein to the vertical connectivity within these isolated wells, without the possibility of examining lateral connections with other samples due to the absence of adjacent wells in the same module.

Additionally, Module 12, which contains only Well 21, faced similar challenges in the connectivity analysis. With only one sample available, it was impossible to establish any form of connectivity, either lateral or vertical, with other reservoir samples. The lack of direct comparison points prevents any conclusions about the fluid connectivity for this specific module.

These limitations highlight the complexity and challenges involved in characterizing fluid connectivity in oil reservoirs, emphasizing the importance of considering the variety of collection methods and the spatial distribution of the wells for a comprehensive and meaningful analysis.

To corroborate the conclusions derived from the PCA analysis, the samples from Well 20 were selected as a reference due to their proven vertical connectivity. This choice is justified by the clear evidence that, although there is a vertical connection within Well 20, there is no lateral interconnection of the fluids with Wells 2 and 7, which belong to the same module. This approach allowed an accurate evaluation of the connectivity dynamics, highlighting the specificity of the interaction between the wells within the same module, while emphasizing the importance of distinguishing between the connectivity types in the reservoir.

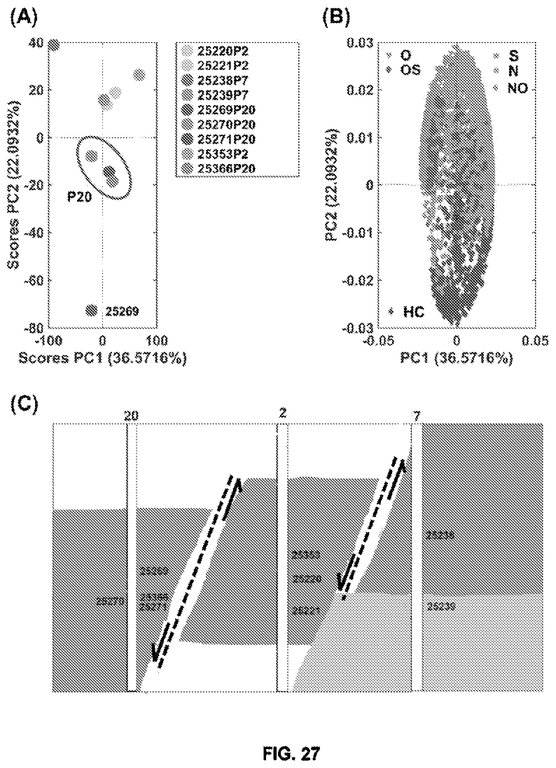

The PCA results related to the samples from module 5 (Wells 2, 7 and 20) are presented in FIG. 27. In the analysis of FIG. 27A, it is possible to note the existence of two groups of similar samples evidenced by the proximity of the scores in both PC1 and PC2. Among them, the group of samples from well 20 (25270, 25271 and 25366) is highlighted in red, which are positioned close together in the graph, indicating a compositional similarity.

These results are consistent, as they corroborate the longitudinal continuity in the sampled interval. However, sample 25269 stands out from the others from well 20, presenting more negative values in PC2, correlating with the HC class in the loadings graph (FIG. 27B). In addition, it is worth noting that this sample was collected at a shallower depth; therefore, this result is suggestive of a greater influence of hydrocarbons (HC class) on the compositional gradient resulting from the depth variations in wells that maintain vertical continuity.

As for the second group of samples, concentrated in the upper right quadrant, with the exception of sample 25238 (well 7), the proximity of the scores also indicates compositional similarity. In addition, the samples from well 20 are separated by PC2, confirming the already highlighted lack of connectivity with the other wells in module 5. Based on these observations, the connection proposal presented in FIG. 27C was developed. In it, well 20 presents vertical continuity in the interval of samples collected from the formation BV, while the lack of connectivity between these samples and the others from wells 2 and 7 can be explained by the presence of flow barriers, such as the geological fault system represented.

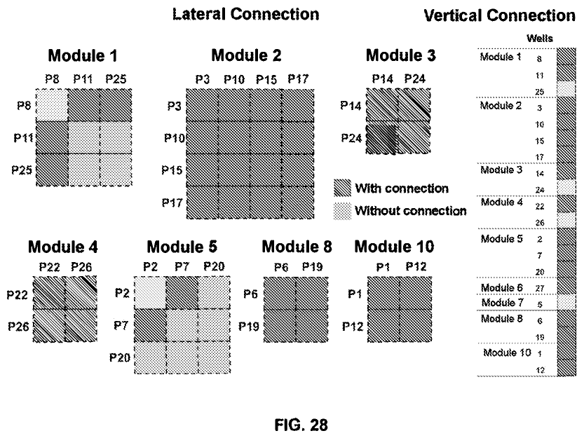

Similarly, based on the PCA scores, proposals for connecting the fluids from the wells in the other modules were developed. The result of the lateral and vertical connection proposal for all the wells in the reservoir is summarized in FIG. 28.

FIG. 28 presents a color scheme to indicate whether or not the reservoir fluids are connected laterally and vertically between the wells in each module. The green squares indicate where there is a connection between the fluids in the wells, either laterally within the same module or vertically through the geological formations. In contrast, the gray squares represent the absence of a connection, suggesting isolated fluid compartments due to the presence of barriers. This visualization allows for a quick and intuitive interpretation of the relations between the wells, which is essential for strategic decision-making in exploration and production.

Therefore, FIG. 28 shows a notable variation in fluid connectivity in the different modules of reservoir X. It is particularly notable that modules 2, 3, 4, 8 and 10 exhibit a complete fluid interconnection between all the wells that make up each module. This suggests that the extraction operations in the wells of these modules are potentially influencing each other due to the extensive fluid communication that allows the fluid movement between the wells of the same module.

On the other hand, in modules 1 and 5, a disconnection is observed, with barriers that prevent full fluid connectivity between all the wells. The differentiated compositions of the fluids extracted in these wells indicate significant heterogeneities in the reservoir, which may be due to variations in the geological composition or in structural features, such as faults or fractures. This finding is fundamental for the production management, since each well may require individual exploration strategies due to its independent connection with the reservoir.

Analyzing vertical connectivity, it is noted that wells 25, 24, 26 and 5 show signs of not being completely vertically connected within their own structures, once again signaling the presence of internal barriers, thus affecting the vertical mobility of the fluids.

In conclusion, the recognition of the vertical and lateral barriers is of vital importance to improve the production management. Understanding the existence and location of these barriers allows the development of more refined oil extraction strategies, adapted to the complex architecture of the reservoir. This detailed discernment of the fluid connectivity is crucial for the advancement of the exploration, and can lead to a more effective operationalization, with cost reduction and increased production, aligning the development of the oil field with sustainable and economically viable practices.

The integrated method for compositional evaluation in oil wells, which will serve as an indicator for lateral and vertical connectivity, offers a holistic and detailed approach to understanding the nature of the fluids extracted from different wells. Whether in the precise identification of the composition, type of operation, type of formation or in determining the connectivity between wells, this method provides valuable insights that can revolutionize the way the oil industry operates, maximizing the efficiency and quality of the extracted oil.

Below are the complementary tables related to the main information about samples and parameters used.

TABLE 5
Sample information regarding the module, well, sample code,
type of operation, geological formation and contamination.

| | | | Operation | Geological | |
|---|---|---|---|---|---|
| Module | Well | Sample | Type | Formation | Contamination |
| 1 | P8 | 25240 | PVT | B | Yes |
| 1 | P8 | 25241 | PVT | B | Yes |
| 1 | P11 | 25250 | PVT | B | |
| 1 | P25 | 25278 | PVT | A | |
| 1 | P25 | 25279 | PVT | B | Yes |
| 1 | P25 | 25280 | PVT | C | |
| 1 | P25 | 25281 | PVT | C | Yes |
| 1 | P8 | 25359 | DST | B | |
| 1 | P25 | 25369 | DST | B | |
| 1 | P25 | 25370 | DST | B | |
| 1 | P25 | 25371 | DST | C | |
| 2 | P3 | 25222 | PVT | B | Yes |
| 2 | P3 | 25223 | PVT | B | Yes |
| 2 | P3 | 25224 | PVT | B | Yes |
| 2 | P3 | 25225 | PVT | C | Yes |
| 2 | P15 | 25258 | PVT | B | |
| 2 | P15 | 25259 | PVT | B | Yes |
| 2 | P17 | 25260 | PVT | B | |
| 2 | P17 | 25261 | PVT | B | |
| 2 | P17 | 25262 | PVT | B | Yes |
| 2 | P3 | 25354 | DST | B | |
| 2 | P3 | 25355 | DST | B | |
| 2 | P10 | 25361 | DST | B | |
| 2 | P15 | 25363 | DST | B | |
| 2 | P17 | 25364 | DST | B | |
| 3 | P14 | 25255 | PVT | A | Yes |
| 3 | P14 | 25256 | PVT | B | Yes |
| 3 | P14 | 25257 | PVT | C | Yes |
| 3 | P24 | 25276 | PVT | B | Yes |
| 3 | P24 | 25277 | PVT | B | Yes |
| 3 | P14 | 25362 | DST | B | |
| 3 | P24 | 25368 | DST | B | |
| 4 | P22 | 25265 | PVT | B | Yes |
| 4 | P22 | 25266 | PVT | C | Yes |
| 4 | P26 | 25282 | PVT | B | Yes |
| 4 | P26 | 25283 | PVT | B | Yes |
| 4 | P26 | 25284 | PVT | C | Yes |
| 4 | P9 | 25360 | DST | B | |
| 5 | P2 | 25220 | PVT | B | Yes |
| 5 | P2 | 25221 | PVT | B | Yes |
| 5 | P7 | 25238 | PVT | B | |
| 5 | P7 | 25239 | PVT | C | |
| 5 | P20 | 25269 | PVT | | |
| 5 | P20 | 25270 | PVT | B | |
| 5 | P20 | 25271 | PVT | B | |
| 5 | P2 | 25353 | DST | B | |
| 5 | P20 | 25366 | DST | B | |
| 6 | P27 | 25285 | PVT | A | |
| 6 | P27 | 25286 | PVT | B | Yes |
| 6 | P27 | 25287 | PVT | C | Yes |
| 6 | P27 | 25288 | PVT | D | Yes |
| 6 | P27 | 25372 | DST | B | |
| 6 | P27 | 25373 | DST | C | |
| 7 | P5 | 25231 | PVT | B | Yes |
| 7 | P5 | 25232 | PVT | B | Yes |
| 7 | P5 | 25233 | PVT | B | Yes |
| 7 | P5 | 25234 | PVT | C | Yes |
| 7 | P5 | 25357 | DST | B | |
| 7 | P5 | 25358 | DST | B | |
| 8 | P6 | 25235 | PVT | B | Yes |
| 8 | P6 | 25236 | PVT | C | Yes |
| 8 | P6 | 25237 | PVT | C | Yes |
| 8 | P19 | 25268 | PVT | A | |
| 8 | P19 | 25365 | DST | B | |
| 9 | P13 | 25254 | PVT | B | Yes |
| 9 | P16 | 25263 | PVT | B | Yes |
| 9 | P16 | 25264 | PVT | B | Yes |
| 10 | P1 | 25213 | PVT | B | Yes |
| 10 | P1 | 25214 | PVT | B | Yes |
| 10 | P1 | 25215 | PVT | B | Yes |
| 10 | P1 | 25216 | PVT | B | Yes |
| 10 | P1 | 25217 | PVT | C | Yes |
| 10 | P1 | 25218 | PVT | C | Yes |
| 10 | P12 | 25251 | PVT | B | Yes |
| 10 | P12 | 25252 | PVT | C | Yes |
| 10 | P1 | 25350 | DST | B | |
| 10 | P1 | 25351 | DST | B | |
| 10 | P1 | 25352 | DST | C | |
| 12 | P21 | 25272 | PVT | B | |
| | P4 | 25227 | PVT | B | Yes |
| | P4 | 25228 | PVT | B | |
| | P4 | 25229 | PVT | B | Yes |
| | P4 | 25230 | PVT | C | Yes |
| | P23 | 25273 | PVT | B | Yes |
| | P23 | 25274 | PVT | C | Yes |
| | P23 | 25275 | PVT | C | |
| | P4 | 25356 | DST | B | |
| | P23 | 25367 | DST | B | |

TABLE 6
Parameters used in the ESI (−) FT-ICR
MS ionization source for sample acquisition.

| Sample Parameters | ESI | Optical Protractor | ESI |
|---|---|---|---|
| | (−) | | (−) |
| Concentration (mg · mL⁻¹) | 0.5 | Time of Flight (ms) | 0.5-0.7 |
| % Basic or acidic reagent | 5% | Frequency (MHz) | 4 |
| Source Parameters | ESI | Radio Frequency Amplitude | 450 |
| | (−) | (Vpp) | |
| Flow (μL · h⁻¹) | 240 | Gas Flow Control (%) | 21 |
| Capillary Voltage (kV) | 4.5 | Analyzer (For Cell) | ESI |
| End Plate Offset | −500 | | (−) |
| Nebulizing Gas (bar/kPa) | 1/100 | Output Transfer Lens (V) | 20 |
| Gas Temperature (° C.) | 200 | Analyzer Input (V) | 8 |
| Capillary Output (V) | −200 | Side Kick | 0 |
| Deflection Plate (V) | −220 | Side Kick Offset (V) | −1.5 |
| Funnel 1 | −150 | Front Trap Plate (V) | −1.5 |
| Skimmer (V) | −45-−80 | Back Trap Plate (V) | −1.5 |
| Funnel Radio Frequency | 140 | Back Trap Plate Quench (V) | −30 |
| Amplitude (Vpp) | | | |
| Collision Energy (V) | 1.5 | Excitation Power (%) | 22 |
| Ion Buildup (s) | 0.005 | Shimming DC Bias | ESI |
| | | | (−) |
| Octopole | ESI | 0° (V) | 1.5 |
| | (−) | 90° (V) | 1.5 |
| Frequency (MHz) | 5 | 180° (V) | 1.5 |
| | | 360° (V) | 1.5 |

TABLE 7
Parameters used in the APPI (+) FT-ICR
MS ionization source for sample acquisition.

| Sample | | Optical | |
|---|---|---|---|
| Parameters | | Protractor | |
| Concentration (mg · mL⁻¹) | 0.5 | Time of Flight (ms) | 0.6-1.2 |
| Source Parameters | | Radio Frequency Amplitude | 370 |
| | | (Vpp) | |
| Flow (μL · h⁻¹) | 400 | Gas Flow Control (%) | 25 |
| Capillary Voltage (kV) | 1.25 | Analyzer (For Cell) | |
| End Plate Offset | −500 | | |
| Nebulizing Gas (bar/kPa) | 1.3/130 | Output Transfer Lens (V) | −20 |
| Gas Temperature (° C.) | 200 | Analyzer Input (V) | −10 |
| Capillary Output (V) | 220 | Side Kick | 0 |
| Deflection Plate (V) | 200 | Side Kick Offset (V) | −3 |
| Funnel 1 | 150 | Front Trap Plate (V) | 1.5 |
| Skimmer (V) | −45-−70 | Back Trap Plate (V) | −1.5 |
| Funnel Radio Frequency | 135 | Back Trap Plate Quench (V) | −30 |
| Amplitude (Vpp) | | | |
| Collision Voltage (V) | −4.5 | Excitation Power (%) | 26 |
| Ion Buildup (s) | 0.003-0.01 | Shimming DC Bias | |
| Octopole | | 0° (V) | 1.528 |
| | | 90° (V) | 1.5 |
| Frequency (MHz) | 5 | 180° (V) | 1.472 |
| | | 360° (V) | 1.5 |

TABLE 8
Molecular formulas with respect to the type
of operation obtained by ESI (−) FT-ICR MS.

| N | N₂ | NO | O | O₂ |
|---|---|---|---|---|
| C₃₅H₂₇N | C₁₈H₂₂N₂ | C₃₄H₄₁NO | C₄₈H₆₈₀ | C₁₁H₁₀O₂ |
| C₃₂H₂₃N | C₂₀H₁₈N₂ | C₃₅H₄₁NO | C₄₃H₅₆₀ | C₁₃H₁₂O₂ |
| C₄₀H₃₇N | C₃₇H₅₈N₂ | C₃₇H₄₇NO | C₅₇H₁₀₀O | C₁₄H₁₀O₂ |
| C₃₃H₂₃N | C₃₈H₅₈N₂ | C₂₉H₃₅NO | C₄₉H₇₀O | C₁₄H₁₄O₂ |
| C₃₉H₃₃N | C₃₉H₆₀N₂ | C₃₂H₃₉NO | C₅₃H₈₀O | C₁₇H₂₀O₂ |
| C₄₇H₅₃N | C₄₅H₇₀N₂ | C₃₆H₄₇NO | C₄₇H₆₆O | C₁₈H₁₄O₂ |
| C₄₁H₃₇N | C₅₈H₇₈N₂ | C₃₇H₄₉NO | C₅₄H₈₄O | C₁₈H₁₆O₂ |
| C₃₈H₃₁N | C₄₃H₄₆N₂ | C₂₃H₂₃NO | C₃₃H₃₆O | C₁₉H₁₆O₂ |
| C₄₀H₃₅N | C₅₃H₇₂N₂ | C₃₅H₄₅NO | C₃₈H₄₈O | C₁₉H₁₈O₂ |
| C₄₂H₃₉N | C₃₅H₂₈N₂ | C₃₆H₄₅NO | C₄₆H₆₆O | C₁₉H₂₂O₂ |
| C₃₆H₂₇N | C₂₂H₁₈N₂ | C₃₄H₄₅NO | C₄₄H₆₀O | C₂₀H₁₆O₂ |
| C₃₁H₂₁N | C₅₂H₇₂N₂ | C₃₃H₄₃NO | C₂₇H₃₀O | C₂₀H₁₈O₂ |
| C₄₀H₃₃N | C₄₉H₆₆N₂ | C₃₂H₄₁NO | C₁₂H₁₄O | C₂₁H₁₆O₂ |
| C₄₇H₄₉N | C₅₂H₇₀N₂ | C₃₈H₅₁NO | C₃₄H₄₀O | C₂₁H₁₈O₂ |
| C₄₉H₅₅N | C₂₃H₂₀N₂ | C₂₁H₁₇NO | C₅₆H₁₀₀O | C₂₁H₂₀O₂ |

TABLE 9
Molecular formulas with respect to the type
of operation obtained by APPI (+) FT-ICR MS.

| HC | N | NO | O | OS | S |
|---|---|---|---|---|---|
| C₃₃H₂₂ | C₄₀H₇₁N | C₄₁H₄₅NO | C₁₄H₁₄O | C₄₆H₈₄OS | C₂₉H₅₆S |
| C₄₁H₃₄ | C₅₄H₉₅N | C₃₇H₃₇NO | C₅₄H₉₈O | C₂₂H₄₄OS | C₂₈H₅₄S |
| C₄₄H₄₀ | C₄₈H₈₅N | C₃₈H₃₉NO | C₂₉H₂₆O | C₄₆H₈₀OS | C₃₀H₅₈S |
| C₃₉H₃₀ | C₃₃H₅₇N | C₃₆H₃₅NO | C₅₇H₈₄O | C₄₈H₈₆OS | C₂₃H₄₄S |
| C₃₆H₂₆ | C₄₉H₈₇N | C₃₉H₄₁NO | C₃₂H₂₈O | C₄₇H₇₈OS | C₃₁H₆₀S |
| C₄₆H₄₀ | C₃₇H₆₅N | C₅₈H₈₇NO | C₄₈H₆₀O | C₄₁H₇₈OS | C₂₆H₅₀S |
| C₄₀H₃₂ | C₃₉H₆₅N | C₃₅H₃₃NO | C₂₃H₁₆O | C₄₄H₈₂OS | C₃₃H₆₄S |
| C₄₇H₄₂ | C₃₄H₄₃N | C₂₄H₂₅NO | C₄₄H₅₂O | C₄₂H₇₈OS | C₂₆H₄₆S |
| C₃₂H₂₂ | C₃₉H₆₇N | C₃₀H₂₅NO | C₃₄H₃₂O | C₃₀H₄₆OS | C₂₇H₅₂S |
| C₄₅H₄₂ | C₅₀H₈₉N | C₃₂H₂₇NO | C₄₃H₄₈O | C₄₆H₇₄OS | C₂₇H₄₈S |
| C₅₅H₆₄ | C₃₅H₆₁N | C₄₇H₇₃NO | C₂₅H₃₀O | C₂₅H₄₀OS | C₃₈H₆₂S |
| C₄₃H₃₄ | C₃₉H₆₉N | C₃₃H₂₉NO | C₅₀H₆₄O | C₁₈H₃₄OS | C₂₈H₃₆S |
| C₅₄H₆₂ | C₃₈H₆₅N | C₃₄H₅₁NO | C₂₈H₂₄O | C₂₀H₃₄OS | C₃₀H₅₂S |
| C₄₂H₃₂ | C₃₅H₅₇N | C₅₀H₇₅NO | C₂₁H₁₈O | C₄₂H₆₈OS | C₂₆H₃₆S |
| C₄₄H₃₆ | C₃₈H₆₃N | C₂₂H₂₅NO | C₃₅H₃₄O | C₄₉H₈₄OS | C₃₄H₄₆S |

TABLE 10
Molecular formulas in relation to geological
formation obtained by ESI (−) FT-ICR MS.

| N | N₂ | NO | O | O₂ |
|---|---|---|---|---|
| C₅₇H₈₇N | C₂₆H₂₆N₂ | C₄₀H₆₉NO | C₃₈H₆₆O | C₁₆H₃₂O₂ |
| C₅₆H₉₁N | C₄₃H₄₆N₂ | C₅₂H₈₃NO | C₄₂H₇₈O | C₁₈H₃₆O₂ |
| C₅₇H₉₃N | C₂₁H₂₂N₂ | C₅₂H₆₁NO | C₄₂H₇₆O | C₁₈H₃₄O₂ |
| C₅₆H₉₃N | C₄₃H₅₀N₂ | C₄₅H₇₅NO | C₃₂H₄₄O | C₁₈H₃₂O₂ |
| C₅₈H₈₇N | C₃₃H₄₆N₂ | C₅₃H₆₃NO | C₄₆H₇₆O | C₂₀H₄₀O₂ |
| C₅₆H₇₇N | C₄₂H₆₂N₂ | C₅₀H₈₃NO | C₃₃H₄₀O | C₁₄H₂₈O₂ |
| C₅₇H₇₉N | C₂₈H₂₄N₂ | C₅₀H₇₉NO | C₄₃H₆₆O | C₂₄H₄₈O₂ |
| C₅₆H₇₅N | C₄₃H₄₈N₂ | C₅₀H₈₁NO | C₄₆H₇₄O | C₁₇H₃₄O₂ |
| C₅₈H₈₁N | C₃₆H₃₄N₂ | C₄₉H₇₇NO | C₄₈H₈₂O | C₂₆H₅₂O₂ |
| C₅₅H₉₅N | C₃₃H₄₈N₂ | C₅₄H₆₅NO | C₅₃H₉₄O | C₂₃H₄₆O₂ |
| C₆₁H₉₇N | C₂₅H₁₈N₂ | C₅₁H₅₉NO | C₂₉H₃₀O | C₁₅H₃₀O₂ |
| C₆₁H₁₀₁N | C₂₈H₄₂N₂ | C₅₁H₇₉NO | C₄₆H₇₀O | C₁₉H₃₈O₂ |
| C₆₁H₉₁N | C₄₂H₄₂N₂ | C₅₀H₅₇NO | C₁₇H₂₂O | C₂₅H₅₀O₂ |
| C₆₁H₈₇N | C₃₀H₄₂N₂ | C₂₆H₁₅NO | C₄₃H₆₂O | C₂₂H₄₄O₂ |
| C₅₉H₈₁N | C₄₆H₆₈N₂ | C₄₂H₃₇NO | C₅₄H₉₄O | C₁₄H₂₂O₂ |

TABLE 11
Molecular formulas in relation to geological
formation obtained by APPI (+) FT-ICR MS.

| HC | N | NO | O | OS | S |
|---|---|---|---|---|---|
| C₃₇H₅₀ | C₅₇H₆₇N | C₆₁H₉₁NO | C₁₉H₂₄O | C₅₂H₈₀OS | C₃₄H₅₀S |
| C₃₆H₄₀ | C₁₇H₂₁N | C₄₄H₄₅NO | C₆₀H₁₀₄O | C₅₄H₁₀₂OS | C₃₃H₄₈S |
| C₃₇H₄₂ | C₆₈H₉₇N | C₅₂H₆₃NO | C₁₈H₂₈O | C₃₀H₄₀OS | C₃₂H₄₆S |
| C₄₂H₅₄ | C₁₈H₂₅N | C₄₂H₇₉NO | C₅₂H₆₀O | C₂₈H₃₈OS | C₃₉H₆₀S |
| C₃₇H₄₀ | C₂₃H₃₇N | C₅₀H₉₃NO | C₂₂H₃₀O | C₅₁H₉₈OS | C₃₈H₅₆S |
| C₃₈H₄₂ | C₇₁H₁₀₉N | C₅₀H₅₉NO | C₂₁H₂₈O | C₅₈H₁₀₂OS | C₂₈H₄₆S |
| C₃₇H₃₈ | C₄₉H₈₉N | C₆₆H₁₁₁NO | C₁₇H₂₀O | C₄₈H₇₂OS | C₃₅H₄₈S |
| C₁₅H₂₄ | C₅₆H₁₀₁N | C₅₄H₇₁NO | C₁₉H₂₆O | C₅₇H₁₀₀OS | C₃₄H₄₆S |
| C₃₆H₃₈ | C₇₂H₁₂₁N | C₅₅H₇₃NO | C₁₉H₂₈O | C₄₃H₆₂OS | C₃₂H₅₂S |
| C₃₆H₃₆ | C₇₀H₁₂₁N | C₆₂H₁₀₉NO | C₂₂H₃₆O | C₅₅H₁₀₄OS | C₃₆H₄₈S |
| C₃₇H₃₆ | C₅₈H₆₉N | C₁₉H₂₅NO | C₁₉H₃₀O | C₄₄H₆₄OS | C₃₈H₅₀S |
| C₄₆H₆₀ | C₇₂H₁₁₁N | C₆₃H₁₀₉NO | C₂₁H₃₄O | C₅₉H₁₀₂OS | C₃₃H₄₂S |
| C₄₂H₄₈ | C₃₂H₂₁N | C₆₁H₁₀₉NO | C₂₁H₃₂O | C₅₁H₇₈OS | C₃₆H₄₆S |
| C₄₁H₄₆ | C₅₅H₆₁N | C₂₉H₁₉NO | C₂₀H₃₀O | C₃₇H₅₀OS | C₃₇H₄₈S |
| C₃₆H₃₄ | C₅₄H₅₉N | C₁₈H₁₇NO | C₁₈H₂₆O | C₅₆H₁₀₂OS | C₄₃H₆₈S |

Based on the teachings of the present invention and its potential impact on the oil industry, the expected advantages are multiple and significant:
1. Greater Accuracy in Analysis: The ability to specifically analyze the polar components in reservoirs and study their variation provides a more detailed view of the composition of the oil, which can result in more accurate analyses than the conventional techniques.
2. Resource Optimization: By determining the connectivity between well fluids through the composition of the polar components, companies can better manage and allocate their resources, avoiding overexploitation of certain areas and maximizing the production.
3. Proactive Decisions: The ability to predict changes in oil composition based on applied techniques and reservoir depth allows companies to make proactive decisions regarding production, refining and commercialization.
4. Cost Savings: A better understanding of the reservoir composition can lead to a reduction in trial and error in exploration and production, significantly saving on operating costs.
5. Development of New Strategies: The invention can pave the way for the development of new exploration and recovery strategies, as well as for innovation in chemical products and processing and refining technologies.
6. Sustainability and Environmental Responsibility: Optimizing production and reducing unnecessary reservoir interventions can reduce the environmental impact of the oil operations, better aligning with the global sustainability goals.
7. Integration with Advanced Technologies: The ability to integrate the findings and analyses of this invention with modern techniques, such as machine learning, offers a holistic and advanced approach to reservoir management and exploration.
8. Quality Assurance: By understanding the factors that influence the composition of the oil, companies can ensure and maintain a consistent quality of the produced oil, better meeting market demands.
9. Improved Geological Understanding: The detailed analysis of the composition of the polar components can provide valuable insights into the geological history and the formation processes of reservoirs.

In summary, the invention offers a robust set of advantages that can transform the way the oil industry approaches exploration, production and reservoir management. Its implications have the potential to improve the operational efficiency, profitability and environmental responsibility of companies in the sector.

1. An integrated method for compositional evaluation in oil wells, comprising:
(a) obtaining crude oil samples through PVT and DST operations;
(b) dissolving the crude oil sample in solvent;
(c) preparing a crude oil sample for ESI (−) analysis by diluting the sample from step (b) in methanol and subsequently adding NH₄OH to the solution diluted with methanol;
(d) preparing a crude oil sample for APPI (+) analysis, wherein the solution of the samples from step (b) is directly injected into a mass spectrometer;
(e) coupling FT-ICR MS equipment to ESI (−) or APPI (+) ionization sources;
(f) calibrating the equipment from step (e);
(g) acquiring spectra in triplicate for each oil sample;
(h) recalibrating the spectra and subsequently assigning molecular formulas; wherein both recalibration of the spectra and assignment of formulas were performed individually for each of the acquisitions;
(i) data alignment and processing of triplicates;
(j) visualization and interpretation of FT-ICR MS data; and
(k) multivariate analysis;
wherein construction of a PLS-DA OPSDA model is obtained through data obtained by ESI (−) and APPI (+) FT-ICR MS.

2. The method according to claim 1, wherein the crude oil samples obtained by the PVT operation undergo stirring and heating to 40° C. prior to analysis.

3. The method according to claim 1, wherein the solvent of step (b) comprises toluene.

4. The method according to claim 1, wherein a final concentration of oil in the analysis solution is 500 ppm in toluene/methanol (50:50) and 5.0% NH₄OH in step (c).

5. The method according to claim 1, wherein the calibration of the equipment in step (e) is performed with a 0.1 μL·mL⁻¹ solution of Sodium Trifluoroacetate calibrant for positive and negative mode, in a m/z range of 150 to 2000.

6. The method according to claim 1, wherein in step (i) the data are aligned and the triplicates are processed for a single spreadsheet.

7. The method according to claim 1, wherein visualization and interpretation of the FT-ICR MS data are performed using graphical tools from petroleomics.

8. The method according to claim 1, wherein the multivariate analysis of step (k) filters the variables resulting from the analysis that presented greater weight in explanation of the model, both for the compositional gradation and for compartmentalization of the reservoir, for PVT and DST.

9. The method according to claim 6, wherein in step (i) the aligned data spreadsheet resulting from the combination of the triplicates was used to obtain information regarding a class present in the samples, molecular formula, DBE, carbon number, monoisotopic abundance and m/z for each of the samples to be analyzed.

10. The method according to claim 1, further comprising analysis of the compositional variation of the polar components in reservoirs; study of the molecular distribution in reservoirs with varied thicknesses; and exploration of the composition of the polar components as molecular indicators of the compartmentalization and lateral and vertical connectivity between the fluids in reservoirs.
